(19) United States
(12) Patent Application Publication
(10) Pub. No.: US 2010/0284933 A1
(43) Pub. Date: Nov. 11, 2010

(54) Biomarkers of Multiple Sclerosis

(75) Inventor: Linda C Burkly, West Newton, MA (US)

Correspondence Address: BIOGEN IDEC / FINNEGAN HENDERSON, LLP, 901 NEW YORK AVENUE, NW, WASHINGTON, DC, 20001-4413, US

(73) Assignee: Biogen Idec MA Inc., Canbridge, MA (US)

(21) Appl. No.: 12/445,598

(22) PCT Filed: Oct. 15, 2007
(86) PCT No.: PCT/US07/81374

§ 371 Date: Apr. 15, 2009

Related U.S. Application Data
(60) Provisional application No. 60/851,957, filed on Oct. 16, 2006.

(51) Int. Cl. A61K 49/14; A61K 49/04; C12Q 1/68; G01N 33/53

(52) U.S. Cl. 424/9.34; 424/9.4; 435/6; 435/7.92; 435/7.1

(57) ABSTRACT

Biomarkers of multiple sclerosis (MS) and of anti-TWEAK/TWEAK-Receptor therapy for MS are described.

### CROSS-REFERENCE TO RELATED APPLICATIONS

This application claims priority to U.S. Application Ser. No. 60/851,957, filed on Oct. 16, 2006. The disclosure of the prior application is considered part of (and is incorporated by reference in) the disclosure of this application.

### SUMMARY

Targeting of the TWEAK signaling pathway, e.g., with a TWEAK/TWEAK receptor (TWEAK/TWEAK-R) blocking agent, can be an effective strategy for treating multiple sclerosis (MS). We found that anti-TWEAK therapies affect the levels of certain biomarkers of MS. For example, peripheral benzodiazepine receptor (PBR) can be used as a key indicator of the efficacy of anti-TWEAK MS therapy.

Therapies targeting the TWEAK signaling pathway can limit or prevent the upregulation of genes associated with MS. For example, therapy with a TWEAK/TWEAK-R blocking agent can counteract or prevent an MS-associated (e.g., MS-induced) upregulation of a gene, e.g., peripheral benzodiazepine receptor (PBR). In some embodiments, the therapy can contribute to amelioration of the symptoms and/or progression of MS. Likewise, in some embodiments, the therapy can downregulatc expression of a gene that is normally upregulated during the course of MS, e.g., in a subject that has been identified as being at risk for developing MS.

According to certain aspects of the invention, a biomarker of MS (e.g., a gene listed in FIG. 3) is evaluated or monitored in a subject. For example, the subject is at risk for, is undergoing a diagnosis for, or has been diagnosed with having MS, and/or is undergoing treatment for MS. In preferred embodiments, the biomarker is PBR. In other preferred embodiments, the MS treatment is a TWEAK/TWEAK-R blocking agent.

In one aspect, the invention features a method of evaluating peripheral benzodiazepine receptor (PBR) in a subject. The method includes evaluating a parameter associated with PBR expression in the subject. For example, the subject has multiple sclerosis (MS) and, for example, has been administered a TWEAK/TWEAK-R blocking agent. In some embodiments, the subject is at risk for MS (e.g., has a genetic predisposition or exhibits one or more symptoms suggestive of MS). The method can be performed as part of making a diagnosis of MS. In other embodiments, the subject has been diagnosed with MS.

In some embodiments, the method includes comparing the parameter in the subject to a reference (e.g., parameter levels in a control, e.g., a subject that does not have MS or an average value of the parameter in a cohort; or parameter levels in the subject prior to commencing treatment with the blocking agent).

In some embodiments, evaluating includes evaluating the parameter prior to treatment with a TWEAK/TWEAK-R blocking agent; and evaluating the parameter after treatment has commenced. For example, the evaluating can be performed about 1 day, about 2 days, about 3 days, about 4 days, about 5 days, about 6 days, about 1 week, about 2 weeks, about 3 weeks, about one month, about 2 months, about 3 months, about 4 months, about 5 months, about 6 months, about 7 months, about 8 months, about 9 months, about 10 months, about 11 months, about 1 year, about 1.5 years later; the duration of treatment can be, e.g., about one month, about 2 months, about 3 months, about 4 months, about 5 months, about 6 months, about 7 months, about 8 months, about 9 months, about 10 months, about 11 months, about 1 year, about 1.5 years, about 2 years, about 2.5 years, about 3 years, about 3.5 years, and so forth after treatment has commenced, or as recommended by a treating physician, or for the duration of the subject's life.

In some embodiments, a decrease in the parameter after treatment has commenced indicates efficacy of the TWEAK/TWEAK-R blocking agent.

In some embodiments, the TWEAK/TWEAK-R blocking agent is selected from the group consisting of: an anti-TWEAK antibody, an anti-TWEAK-R (e.g., Fn14) antibody, and a soluble form of the TWEAK receptor.

In some embodiments of the method, the evaluating is performed in vivo. In some embodiments, the evaluating comprises a PET scan or MRI. In some preferred embodiments, a region of the spinal cord or brain of the subject is evaluated. In some embodiments, the evaluating comprises quantitative or qualitative assessment of PBR nucleic acid (e.g., mRNA or cDNA) levels. In some embodiments, the evaluating comprises quantitative or qualitative assessment of PBR protein levels. In some embodiments, the parameter reflects the amount of PBR binding agent that binds to PBR protein or PBR nucleic acid (e.g., and correlates with the level of PBR protein or nucleic acid present). In some embodiments, the evaluating is performed by administering a PBR binding agent to the subject. In preferred embodiments, the agent is a PBR ligand (e.g., PK 11195) or an anti-PBR antibody. In some embodiments, the binding agent is labeled with a detectable label (e.g., fluorescent label or radiolabel, e.g., ¹¹C).

In other embodiments, the evaluating is performed in vitro on a sample (e.g., blood, serum, plasma, cerebrospinal fluid, biopsy, or urine sample) obtained from the subject. In some embodiments, the evaluating comprises quantitative or qualitative assessment of PBR nucleic acid (e.g., mRNA or cDNA) levels. In some embodiments, the evaluating comprises quantitative or qualitative assessment of PBR protein levels. In preferred embodiments, evaluating is performed by a technique selected from the group consisting of: RT-PCR, Northern blot, ELISA, Western Blot, flow cytometry, and autoradiography. In some embodiments, the evaluating is performed by contacting a PBR binding agent to the sample. In preferred embodiments, the agent is a PBR ligand (e.g., PK 11195) or an anti-PBR antibody. In some preferred embodiments, the binding agent is labeled with a detectable label (e.g., fluorescent label or radiolabel, e.g., ¹¹C, ³H).

In some embodiments, the subject is human.

In another aspect, the invention features a method of evaluating a subject who is being treated for MS. The method includes monitoring a parameter associated with PBR expression in a subject; and providing a TWEAK/TWEAK-R blocking agent to ameliorate MS to the subject.

In some embodiments, the monitoring comprises evaluating a parameter associated with PBR expression from a subject at least two instances separated by at least 24 hours.

In some embodiments, the method includes comparing the parameter in the subject to a reference (e.g., parameter levels in a control, e.g., a subject that does not have MS or an average value of the parameter in a cohort; or parameter levels in the subject prior to commencing treatment with the blocking agent).

In some embodiments, monitoring comprises evaluating the parameter prior to treatment with a TWEAK/TWEAK-R blocking agent; and evaluating the parameter after treatment has commenced, e.g., about 1 day, about 2 days, about 3 days, about 4 days, about 5 days, about 6 days, about 1 week, about 2 weeks, about 3 weeks, about one month, about 2 months, about 3 months, about 4 months, about 5 months, about 6 months, about 7 months, about 8 months, about 9 months, about 10 months, about 11 months, about 1 year, about 1.5 years later; the duration of treatment can be, e.g., about one month, about 2 months, about 3 months, about 4 months, about 5 months, about 6 months, about 7 months, about 8 months, about 9 months, about 10 months, about 11 months, about 1 year, about 1.5 years, about 2 years, about 2.5 years, about 3 years, about 3.5 years, and so forth after treatment has commenced, as recommended by a treating physician, or for the duration of the subject's life.

In some embodiments, a decrease in the parameter after treatment has commenced indicates efficacy of the TWEAKJTWEAK-R blocking agent. In some embodiments, the TWEAK/TWEAK-R blocking agent is selected from the group consisting of: an anti-TWEAK antibody, an anti-TWEAK-R (e.g., Fn14) antibody, and a soluble form of the TWEAK receptor.

In some embodiments, the monitoring is performed in vivo. In some embodiments, the parameter reflects the amount of PBR binding agent that binds to PBR protein or PBR nucleic acid. In some embodiments, the monitoring comprises quantitative or qualitative assessment of PBR nucleic acid (e.g., mRNA or cDNA) levels. In other embodiments, the monitoring comprises quantitative or qualitative assessment of PBR protein levels. In preferred embodiments, the monitoring comprises a PET scan or MRI. In more preferred embodiments, a region of the spinal cord or brain of the subject is evaluated. In some embodiments, the monitoring is performed by administering a PBR binding agent to the subject. In preferred embodiments, the agent is a PBR ligand (e.g., PK 11195) or an anti-PBR antibody. In more preferred embodiments, the binding agent is labeled with a detectable label (e.g., fluorescent label or radiolabel, e.g., ¹¹C).

In other embodiments, the monitoring is performed in vitro on a sample (e.g., blood, serum, plasma, cerebrospinal fluid, biopsy, or urine sample) obtained from the subject. In some embodiments, the monitoring comprises quantitative or qualitative assessment of PBR nucleic acid (e.g., mRNA or cDNA) levels. In other embodiments, the monitoring comprises quantitative or qualitative assessment of PBR protein levels. In some embodiments, monitoring is performed by a technique selected from the group consisting of: RT-PCR, Northern blot, ELISA, Western Blot, flow cytometry, and autoradiography. In some embodiments, the monitoring is performed by contacting a PBR binding agent to the sample. In preferred embodiments, the agent is a PBR ligand (e.g., PK 11195) or an anti-PBR antibody. In more preferred embodithents, the binding agent is labeled with a detectable label (e.g., fluorescent label or radiolabel, e.g., ¹¹C, ³H).

In some embodiments, the subject is human.

In another aspect, the invention features a method of evaluating a subject who is being treated for MS. The method includes treating a subject with a TWEAK/TWEAK-R blocking agent for MS; before, during, or after treatment with the blocking agent, monitoring a parameter associated with PBR expression in the subject; and comparing results of the evaluation to a reference to provide an assessment of the subject.

In some embodiments, the reference is obtained by a corresponding evaluation of the subject prior to commencing treatment with the blocking agent.

In some embodiments, the reference is parameter levels in a control, e.g., a subject that does not have MS or an average value of the parameter in a cohort; or parameter levels in the subject prior to commencing treatment with the blocking agent.

In some embodiments, monitoring comprises evaluating the parameter prior to treatment with a TWEAK/TWEAK-R blocking agent; and evaluating the parameter after treatment has commenced, e.g., about 1 day, about 2 days, about 3 days, about 4 days, about 5 days, about 6 days, about 1 week, about 2 weeks, about 3 weeks, about one month, about 2 months, about 3 months, about 4 months, about 5 months, about 6 months, about 7 months, about 8 months, about 9 months, about 10 months, about 11 months, about 1 year, about 1.5 years later; the duration of treatment can be, e.g., about one month, about 2 months, about 3 months, about 4 months, about 5 months, about 6 months, about 7 months, about 8 months, about 9 months, about 10 months, about 11 months, about 1 year, about 1.5 years, about 2 years, about 2.5 years, about 3 years, about 3.5 years, and so forth after treatment has commenced, as recommended by a treating physician, or for the duration of the subject's life.

In some embodiments, a decrease in the parameter after treatment has commenced indicates efficacy of the TWEAK/TWEAK-R blocking agent.

In some embodiments, the TWEAK/TWEAK-R blocking agent is selected from the group consisting of: an anti-TWEAK antibody, an anti-TWEAK-R (e.g., Fn14) antibody, and a soluble form of the TWEAK receptor.

In some embodiments, the monitoring is performed in vivo. In some embodiments, the parameter reflects the amount of PBR binding agent that binds to PBR protein or PBR nucleic acid. In some embodiments, the monitoring comprises quantitative or qualitative assessment of PBR nucleic acid (e.g., mRNA or cDNA) levels. In other embodiments, the monitoring comprises quantitative or qualitative assessment of PBR protein levels. In preferred embodiments, the monitoring comprises a PET scan or MRI. In more preferred embodiments, a region of the spinal cord or brain of the subject is evaluated. In some embodiments, the monitoring is performed by administering a PBR binding agent to the subject. In preferred embodiments, the agent is a PBR ligand (e.g., PK 11195) or an anti-PBR antibody. In more preferred embodiments, the binding agent is labeled with a detectable label (e.g., fluorescent label or radiolabel, e.g., ¹¹C).

In other embodiments, the monitoring is performed in vitro on a sample (e.g., blood, serum, plasma, cerebrospinal fluid, biopsy, or urine sample) obtained from the subject. In some embodiments, the monitoring comprises quantitative or qualitative assessment of PBR nucleic acid (e.g., mRNA or cDNA) levels. In other embodiments, the monitoring comprises quantitative or qualitative assessment of PBR protein levels. In some embodiments, monitoring is performed by a technique selected from the group consisting of: RT-PCR, Northern blot, ELISA, Western Blot, flow cytometry, and autoradiography. In some embodiments, the monitoring is performed by contacting a PBR binding agent to the sample. In preferred embodiments, the agent is a PBR ligand (e.g., PK 11195) or an anti-PBR antibody. In more preferred embodiments, the binding agent is labeled with a detectable label (e.g., fluorescent label or radiolabel, e.g., ¹¹C, ³H).

In some embodiments, the subject is human.

In a most preferred embodiment, the invention features a method of evaluating a subject who is being treated for MS, the method includes treating a subject with an anti-TWEAK antibody for MS; during treatment with the antibody, in vivo monitoring PBR protein levels in the subject, wherein the monitoring comprises administering ¹¹C-labeled PK 11195 to the subject and imaging the subject by use of a PET scan to detect PK 11195 binding to PBR protein; and comparing results of the monitoring to a corresponding monitoring of the subject prior to commencing treatment with the antibody, wherein a decrease in PK 11195 binding to PBR protein after treatment has commenced indicates efficacy of the antibody.

In another aspect, the invention features a method of altering the dosage of a TWEAK/TWEAK-R blocking agent. The method includes evaluating a parameter associated with PBR expression in a subject, wherein the subject has MS and is being treated with a TWEAK/TWEAK-R blocking agent.

In some embodiments, the evaluating includes evaluating a parameter of PBR expression in the subject prior to commencing treatment with the TWEAK/TWEAK-R blocking agent; and evaluating the parameter in the subject after commencing treatment with the TWEAK/TWEAK-R blocking agent, wherein absence of a decrease in the parameter after treatment has commenced indicates that the dosage of the therapy can be altered (e.g., increased or decreased).

In some embodiments, the method further includes making a treatment decision.

In some embodiments, evaluating includes evaluating a parameter of PBR expression in the subject prior to commencing treatment with the TWEAK/TWEAK-R blocking agent; and evaluating the parameter in the subject after commencing treatment with the TWEAK/TWEAK-R blocking agent, wherein absence of a decrease in the parameter after treatment has commenced indicates that a second therapy can be administered to the subject (e.g., second therapy (e.g., another treatment for MS) alone or in combination with the TWEAK/TWEAK R blocking agent).

In some embodiments, evaluating includes evaluating the parameter prior to treatment with a TWEAK/TWEAK-R blocking agent; and evaluating the parameter e.g., about 1 day, about 2 days, about 3 days, about 4 days, about 5 days, about 6 days, about 1 week, about 2 weeks, about 3 weeks, about one month, about 2 months, about 3 months, about 4 months, about 5 months, about 6 months, about 7 months, about 8 months, about 9 months, about 10 months, about 11 months, about 1 year, about 1.5 years after treatment has commenced.

In some embodiments, a decrease in the parameter after treatment has commenced indicates efficacy of the TWEAK/TWEAK-R blocking agent.

In some embodiments, the TWEAK/TWEAK-R blocking agent is selected from the group consisting of: an anti-TWEAK antibody, an anti-TWEAK-R (e.g., Fn14) antibody, and a soluble form of the TWEAK receptor.

In some embodiments of the method, the evaluating is performed in vivo. In some embodiments, the evaluating comprises a PET scan or MRI. In some preferred embodiments, a region of the spinal cord or brain of the subject is evaluated. In some embodiments, the evaluating comprises quantitative or qualitative assessment of PBR nucleic acid (e.g., mRNA or cDNA) levels. In some embodiments, the evaluating comprises quantitative or qualitative assessment of PBR protein levels. In some embodiments, the parameter reflects the amount of PBR binding agent that binds to PBR protein or PBR nucleic acid. In some embodiments, the evaluating is performed by administering a PBR binding agent to the subject. In preferred embodiments, the agent is a PBR ligand (e.g., PK 11195) or an anti-PBR antibody. In some embodiments, the binding agent is labeled with a detectable label (e.g., fluorescent label or radiolabel, e.g., ¹¹C).

In other embodiments, the evaluating is performed in vitro on a sample (e.g., blood, serum, plasma, cerebrospinal fluid, biopsy, or urine sample) obtained from the subject. In some embodiments, the evaluating comprises quantitative or qualitative assessment of PBR nucleic acid (e.g., mRNA or cDNA) levels. In some embodiments, the evaluating comprises quantitative or qualitative assessment of PBR protein levels. In preferred embodiments, evaluating is performed by a technique selected from the group consisting of: RT-PCR, Northern blot, ELISA, Western Blot, flow cytometry, and autoradiography. In some embodiments, the evaluating is performed by contacting a PBR binding agent to the sample. In preferred embodiments, the agent is a PBR ligand (e.g., PK 11195) or an anti-PBR antibody. In some preferred embodiments, the binding agent is labeled with a detectable label (e.g., fluorescent label or radiolabel, e.g., ¹¹C, ³H).

In some embodiments, the subject is human.

In another aspect, the invention features a method of evaluating an MS therapeutic. The method includes evaluating a parameter associated with PBR expression in a subject, wherein the subject has MS or experimental autoimmune encephalomyelitis (EAE).

In some embodiments, the therapeutic comprises a TWEAK/TWEAK-R blocking agent.

In some embodiments, the method also includes evaluating TWEAK pathway activity in the subject.

In some embodiments, the evaluating includes evaluating a parameter of PBR expression in the subject prior to commencing treatment with the therapeutic; evaluating a parameter of PBR expression in the subject after commencing treatment with the therapeutic; wherein absence of a decrease in the parameter after treatment has commenced indicates that the therapeutic is not effective for treating MS or that the dosage of the therapeutic should be altered.

In some embodiments, the evaluating includes evaluating a parameter of PBR expression in the subject after commencing treatment with the therapeutic; and obtaining a reference value for the parameter, wherein a reference value lower than the parameter indicates that the therapeutic is not effective for treating MS or that the dosage of the therapeutic should be altered.

In some embodiments, the reference value is parameter levels (e.g., mRNA, cDNA, or protein levels) in a control, e.g., a subject that does not have MS or an average value of the parameter in a cohort; or parameter levels in the subject prior to commencing treatment with the blocking agent.

In some embodiments, evaluating includes evaluating the parameter prior to treatment with a TWEAK/TWEAK-R blocking agent; and evaluating the parameter after treatment has commenced, e.g., about 1 day, about 2 days, about 3 days, about 4 days, about 5 days, about 6 days, about 1 week, about 2 weeks, about 3 weeks, about one month, about 2 months, about 3 months, about 4 months, about 5 months, about 6 months, about 7 months, about 8 months, about 9 months, about 10 months, about 11 months, about 1 year, about 1.5 years after commencement of treatment.

In some embodiments, a decrease in the parameter after treatment has commenced indicates efficacy of the TWEAK/TWEAK-R blocking agent.

In some embodiments, the TWEAK/TWEAK-R blocking agent is selected from the group consisting of: an anti-TWEAK antibody, an anti-TWEAK-R (e.g., Fn14) antibody, and a soluble form of the TWEAK receptor.

In some embodiments of the method, the evaluating is performed in vivo. In some embodiments, the evaluating comprises a PET scan or MRI. In some preferred embodiments, a region of the spinal cord or brain of the subject is evaluated. In some embodiments, the evaluating comprises quantitative or qualitative assessment of PBR nucleic acid (e.g., mRNA or cDNA) levels. In some embodiments, the evaluating comprises quantitative or qualitative assessment of PBR protein levels. In some embodiments, the parameter reflects the amount of PBR binding agent that binds to PBR protein or PBR nucleic acid. In some embodiments, the evaluating is performed by administering a PBR binding agent to the subject. In preferred embodiments, the agent is a PBR ligand (e.g., PK 11195) or an anti-PBR antibody. In some embodiments, the binding agent is labeled with a detectable label (e.g., fluorescent label or radiolabel, e.g., ¹¹C).

In other embodiments, the evaluating is performed in vitro on a sample (e.g., blood, serum, plasma, cerebrospinal fluid, biopsy, or urine sample) obtained from the subject. In some embodiments, the evaluating comprises quantitative or qualitative assessment of PBR nucleic acid (e.g., mRNA or cDNA) levels. In some embodiments, the evaluating comprises quantitative or qualitative assessment of PBR protein levels. In preferred embodiments, evaluating is performed by a technique selected from the group consisting of: RT-PCR, Northern blot, ELISA, Western Blot, flow cytometry, and autoradiography. In some embodiments, the evaluating is performed by contacting a PBR binding agent to the sample. In preferred embodiments, the agent is a PBR ligand (e.g., PK 11195) or an anti-PBR antibody. In some preferred embodiments, the binding agent is labeled with a detectable label (e.g., fluorescent label or radiolabel, e.g., ¹¹C, ³H).

In some embodiments, the subject is human. In other embodiments, the subject is a mouse.

The methods that are described herein with respect to PBR proteins and nucleic acid can also be implemented with each other gene listed in FIG. 3 (e.g., C-type lectin, superfamily member 10, interleukin 1 receptor antagonist, C-type lectin, superfamily member 8, macrophage scavenger receptor 1, Fc receptor, IgG, low affinity lib, C-type lectin, superfamily member 9, interleukin 1 beta, mannose receptor, C type 1, benzodiazepine receptor, peripheral, lectin, galactose binding, soluble 3, CD14 antigen, Fc receptor, IgG, low affinity IIb, toll-like receptor 2, cathepsin C, lysozyme, colony stimulating factor 2 receptor, beta 1, low-affinity, colony stimulating factor 2 receptor, beta 2, low-affinity, toll-like receptor 4, histocompatibility 2, class II antigen E beta, integrin beta 2, CD86 antigen, histocompatibility 2, class II antigen A, beta 1, ferritin light chain 2, toll-like receptor 8, C-type lectin, superfamily member 5, C-type lectin, superfamily member 6, Fc receptor, IgG, low affinity III, toll-like receptor 7, Fc receptor, IgG, high affinity I, histocompatibility 2, class II antigen A, alpha, lipocalin 2, C-type lectin, superfamily member 12, cathepsin H, and, cathepsin Z). The term “treating” refers to administering a therapy in an amount, manner, and/or mode effective to improve or prevent a condition, symptom, or parameter associated with MS.

In another aspect, the disclosure features a method of evaluating a subject having, or suspected of having, multiple sclerosis (MS). The method includes: evaluating the subject to obtain a parameter associated with one or more genes whose levels are altered by a TWEAK blocking agent, for example, one or more genes listed in FIG. 3.

All publications, patent applications, patents, and other references mentioned herein are incorporated by reference in their entirety. In the case of conflict, the present specification, including definitions, controls. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a line graph showing the effects of anti-TWEAK therapy on mean clinical score in an EAE model of disease. Mice were treated on day 9, 13, 17, and 21 after the induction of EAE. AB.G11 is a hamster anti-TWEAK antibody; P2D10 is a murine anti-TWEAK antibody. Ha 4/8 and P1.17 are isotype-matched controls for AB.G11 and P2D10, respectively.

FIGS. 2A and 2B are bar graphs depicting the percentage of pathway qualifiers affected during EAE. FIG. 2A shows the percentage of pathway qualifiers affected during EAE. FIG. 2B shows the percentage of the pathway qualifiers from FIG. 2A for which the upregulation seen in FIG. 2A is counteracted by anti-TWEAK treatment on day 17.

FIG. 3 is a table listing genes associated with microglial activation whose expression is upregulated during the course of EAE and whose upregulation in EAE is counteracted by treatment with anti-TWEAK antibody.

FIG. 4 is a graph showing the levels of TWEAK in the serum of patients with relapsing remitting MS (RRMS), primary progressive MS (PPMS), secondary progressibe MS (SPMS), or in controls.

### DETAILED DESCRIPTION

TWEAK levels can be elevated in patients with MS. Therapy with a TWEAK/TWEAK-R blocking agent can be an effective treatment for alleviating clinical symptoms or progression of MS. This effect is mediated, at least in part, by decreasing microglial and macrophage activation.

We have identified genes whose expression levels are upregulated in spinal cords during the course of experimental autoimmune encephalomyelitis (EAE) in mice, which serves as a model of many aspects of MS. Microglia- and macrophage-associated genes were identified as being upregulated during this disease. Thus, the expression level of one or more of these genes (or the level and/or activity of the proteins encoded by these genes) during the course of MS can serve as a biomarker of disease progression.

Significantly, treatment of EAE mice with anti-TWEAK antibody therapy limits the upregulation of these genes. The expression level of one or more of these genes during the course of treatment with a TWEAK/TWEAK-R blocking agent (e.g., anti-TWEAK antibody, anti-TWEAK receptor antibody, or soluble form of the TWEAK receptor), or other MS therapeutic, can serve as a biomarker of the treatment, e.g., of the efficacy of the treatment.

The levels (e.g., nucleic acid or protein) of a biomarker can be measured in a subject, e.g., in a sample obtained from the subject (e.g., blood, serum, plasma, cerebrospinal fluid, biopsy, or urine sample) or by measuring the levels of the biomarker in vivo in a subject. For example, an agent (e.g., an agent that can be detected and measured) that binds to a biomarker (e.g., nucleic acid or protein) can be contacted in vitro to a sample from the subject and the levels of the agent bound to the biomarker can be measured. As another example, an agent (e.g., an agent that can be detected and measured) that binds to a biomarker (e.g., nucleic acid or protein) can be administered to a subject and the levels of the agent bound to the biomarker, e.g., in a region of interest (such as the central nervous system (CNS), e.g., brain or spinal cord), can be measured.

Multiple Sclerosis

Multiple sclerosis (MS) is a central nervous system disease that is characterized by inflammation and loss of myelin sheaths. MS is generally considered to be an autoimmune disease.

In MS, the immune system mistakenly destroys the cells that produce the myelin sheath which surrounds nerves in the brain and spinal cord, causing inflammation and injury to the sheath and ultimately to the nerves that it surrounds. Myelin becomes inflamed and swollen and detaches from the fibers. The detached myelin may eventually be destroyed. Sclerosed patches of scar tissue form over the fibers. When nerve impulses reach a damaged area, some impulses are blocked or delayed from traveling to or from the brain. Ultimately, this process leads to degeneration of the nerves themselves, which likely accounts for the permanent disabilities that may develop in MS.

Patients having MS may be identified by criteria establishing a diagnosis of clinically definite MS as defined by the workshop on the diagnosis of MS (Poser et al., Ann. Neurol. (1983) 13:227). Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid.

MS Occurs in Four Main Patterns:

Relapsing remitting. This type of MS is characterized by clearly defined flare-ups, followed by periods of remission. The flare-ups typically appear suddenly, last a few weeks or months, and then gradually disappear. Most people with MS have this form at the time of diagnosis.

Primary progressive. Subjects with this less common form of MS experience a gradual decline, without periods of remission. Subjects with this form of MS are usually older than 40 when symptoms begin.

Secondary progressive. More than half the people with relapsing remitting MS eventually enter a stage of continuous deterioration referred to as secondary progressive MS. Sudden relapses may occur, superimposed upon the continuous deterioration that characterizes this type of MS.

Progressive relapsing. This is primary progressive MS with the addition of sudden episodes of new symptoms or worsened existing ones. This form is relatively rare.

Therapies for the treatment of MS progression and/or amelioration of disease symptoms include: TWEAK/TWEAK-R blocking agents (such as antibodies (e.g., antagonist antibodies) or soluble forms of the TWEAK receptor), beta interferons (such as beta-1a and beta-1b interferons), glatiramer, mitoxantrone, cyclophosphamide, corticosteroids, baclofen, tizanidine, amantadine, and modafinil.

Effective treatment of multiple sclerosis may be examined in several different ways. The following parameters can be used to gauge effectiveness of treatment. Three main criteria are used: EDSS (extended disability status scale), appearance of exacerbations or MRI (magnetic resonance imaging). The EDSS is a means to grade clinical impairment due to MS (Kurtzke (1983) Neurology 33:1444). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of in EDSS indicates an effective treatment (Kurtzke (1994) Ann. Neurol. 36:573-79).

An exemplary animal model for MS is the experimental autoimmune encephalomyelitis (EAE) rodent (e.g., mouse) model, e.g., as described in Tuohy et al. (J. Immunol. (1988) 141: 1126-1130), Sobel et al. (J. Immunol. (1984) 132: 2393-2401), and Traugott (Cell Immunol. (1989) 119: 114-129). EAE mice can be employed to test the efficacy of a potential MS therapy. For example, a potential therapy can be administered prior to EAE induction, or during EAE development, or after disease onset. The mice are evaluated for characteristic criteria to determine the efficacy of a potential MS therapy.

Peripheral Benzodiazepine Receptor

Benzodiazepines are used clinically as muscle relaxants, anticonvulsants, anxiolytics, and sedative-hypnotics. Benzodiazepines can bind to two types of receptors: central benzodiazepine receptors (CBRs) and peripheral benzodiazepines receptors (PBRs).

PBRs are composed of at least three subunits: the binding site for isoquinolines, with a molecular mass of 18 kDa; the voltage-dependent anion channel (VDAC), with a molecular mass of 32 kDa, which binds benzodiazepines; and the adenine nucleotide carrier, with a molecular mass of 30 kDa, which also binds benzodiazepines. Although isoquinolines can bind to the 18-kDa subunit alone, PBR-specific benzodiazepines require the interaction of all three subunits for binding. Isoquinolines that bind specifically to PBRs interact specifically with the 18-kDa subunit, whereas PBR-specific ligands bind to a site consisting of both VDAC and the 18-kDa PBR subunits.

PBRs are found not only in peripheral tissue but also in non-neuronal brain tissue. PBR densities are high in steroidogenic tissues, in particular in the adrenal gland. PBR densities in tissues such as the kidney, heart, testis, ovary, and uterus are approximately five times as low as that in the adrenal but are still one order of magnitude higher than in other tissues such as the brain. For a review on PBR, see Gavish et al. (1999) Pharmacological Reviews 51:629-650.

The cDNA for the 850-nucleotide PBR mRNA has been cloned for a number of species, including humans, rat, mouse, and cows. The genes for human and rat PBR have been partially cloned and characterized. This approximately 13-kbp gene was found as a single copy in the human genome and located on chromosome 22 in the 22q13.31 band. This gene is composed of four exons, with the first exon and half of the fourth exon being untranslated. This gene has one transcription initiation site. An alternatively spliced human PBR mRNA has been reported. Exemplary PBR sequences include human PBR (cDNA sequence of NCBI accession no. M36035; amino acid sequence AAA03652); rat PBR (the cDNA sequence NM_{—}012515; amino acid sequence NP_{—}036647); and mouse PBR (cDNA sequence L17306; amino acid sequence AAA20127). Probes that bind to (e.g., are complementary to) a PBR nucleic acid (e.g., a sequence disclosed herein) can also be used to measure PBR levels, as described herein.

PBR ligands include the following. Protoporphyrin IX is an endogenous ligand for PBRs. The benzodiazepine Ro 5-4864 (4′-chlorodiazepam) and the non-benzodiazepine ligand PK 11195 (an isoquinoline carboxamide derivative, [1-(2-chlorophenyl-N-methyl-N-(1-methylpropyl)-3-isoquinoline carboxamide]) bind to PBRs with high affinity but to CBRs with negligible affinity. FG1N-1 (2-aryl-3-indoleacetamide) binds with high affinity to PBRs but not to CBRs. PK 11195 is commercially available (e.g., from Tocris, Baldwin, Mo., USA).

PBR has been used as a sensitive marker to visualize and measure glial cell activation associated with various forms of brain injury and inflammation in vivo. PBR ligands can be used for in vivo imaging and detection of PBR. For example, PK 11195 can be labeled with a radiolabel such as ¹¹C for PET imaging. See Debruyne et al. (2003) European J. Neural. 10: 257-264; and Banati et al. (2000) Brain 123: 2321-2337.

The PBR ligands can also be used to measure PBR protein levels in samples obtained from a subject (e.g., blood, serum, plasma, cerebrospinal fluid, or urine) in in vitro assays, such as autoradiography, flow cytometry, and ELISA studies. The ligands can be labeled with a radiolabel, fluorescent label, or other detectable label, or can be detected by binding of another agent that itself is labeled.

Antibodies to PBR can also be used to measure PBR protein levels. The antibodies can be used for in vivo imaging, and can also be used in in vitro assays, such as ELISA, Western blotting, and flow cytometry. The antibodies can be labeled with a radiolabel, fluorescent label, or other detectable label, or can be detected by binding of another agent that itself is labeled, e.g., a labeled secondary antibody. Commercial antibodies are available, e.g., from Trevigen, AbCam, R&D Systems, and Santa Cruz Biotechnology, Inc.

Gene Expression Profiling

Biomarkers of MS can be identified by comparing expression levels of genes (e.g., mRNA levels) between two samples, e.g., from a control animal and a test animal, e.g., an animal with EAE (e.g., a mouse) or with MS (e.g., a human); an animal with EAE (or MS) before and after treatment, e.g., with an anti-TWEAK pathway therapy.

DNA array technology allows for the simultaneous measurement of the expression level of thousands of genes in a single experiment. Each array consists of a solid support (usually nylon or glass) in which cDNA or oligonucleotides (targets) are spotted in a known pattern. Fluorescent or radioactive genetic material (probes) derived from mRNA from a source of interest (e.g., a control or test animal) are hybridized to the complementary DNA on the array. The radioactive or fluorescence emissions from the specifically bound probe are detected using an appropriate scanner, giving a quantitative estimate of each gene expression. Ultimately, these signals represent the amounts of mRNA originally present in the cell.

Two main types of DNA arrays are used. The first uses arrays of cDNA clones robotically spotted on a solid surface in the form of polymerase chain reaction (PCR) products. Several versions exist depending on the type of support (nylon, glass) and the type of target labeling (radioactivity, colorimetry, fluorescence). Glass-based full-length cDNA arrays are widely used, in which the DNA probes are labeled by incorporation of fluorescently tagged nucleotides. Typically, two probes are hybridized on a single array (test and control probes), each labeled with two different fluorophores. The expression of a gene in a test sample/animal is then expressed as a relative ratio with respect to the control sample/animal. In case of nylon arrays, an automatic gridder prints PCR amplified cDNA to positively charged nylon membranes, and RNA probes are labeled with P³³ or P³²-dCTP during a reverse transcription reaction.

The second implementation uses arrays of oligonucleotides either directly synthesized in situ on a support or robotically spotted. Probe design requires knowledge of the gene sequences. Their length (oligonucleotides of 20-80 bp) allows for differential detection of members of gene families or alternative transcripts not distinguishable with full-length cDNA arrays. This technology uses a different system to label the probe. Message RNA is converted in biotinylated complementary RNA before being hybridized to the array. Each sample is hybridized to a different array and every array is incubated with an avidin-conjugated fluorophore.

The comparison of independent samples (e.g., control and test samples) is often performed using filtering rules based on arbitrarily assigned fold-difference criteria. Despite the good results yielded with this method, it is possible that the application of a simple fold-based rule leads to false-positive results. Classic statistical techniques can be adopted to test the significance of the observed differences. For example, if two independent samples are compared, a standard t test is appropriate. The genes in the array can be ranked according to increasing P values and an appropriate threshold can be chosen depending on the designated percentage of false-positives (e.g., of 1%, 2%, 5%, 8%, 10% and so forth). A paired t test can be used to assess the significance of the differences. More complex experimental situations may involve the comparison of multiple samples.

DNA arrays deliver several thousands of measurements per experiment. Although genes that display extreme expression changes between samples may require specific analysis, the true strength of high-throughput experiments in revealing the complexity of tumor/host relation derives from the mathematical identification of expression patterns (called “signatures”) within profiling data. In the context of gene expression studies, this involves finding similar gene expression patterns by comparing profiles. Dedicated software developed for this task includes the “unsupervised” and “supervised” varieties. Unsupervised methods (e.g., cluster analysis, self-organizing map (SOM), principal component analysis (PCA)) define classes without any a priori intervention on data, which are organized by clustering genes and/or samples simply according to similarities in their expression profiles. The resulting sample classification often correlates with a general characteristic of the sample as defined by large sets of genes and not necessarily with the particular feature of interest, generally identified by a smaller set of genes. By defining relevant classes before analysis, supervised techniques (e.g., support vector machines, weighted votes, and neural networks) bypass this issue. These algorithms incorporate external information related to samples studied to identify the optimal set of genes that best discriminate between experimental samples.

Depending on the way in which the data are clustered, hierarchical and nonhierarchical clustering can be distinguished. Hierarchical clustering allows detection of higher-order relationships between clusters of profiles. By contrast, the majority of nonhierarchical classification techniques work by allocating gene expression profiles to a predefined number of clusters (supervised methods).

Unsupervised neural networks, and in particular SOM, provide a more robust and accurate approach to the clustering of large sets of data. They are robust with respect to noise, and they are generally independent of the shape of the data distribution. Another advantage of neural networks using SOM is the high performance displayed with large sets of data. Initially, genes are randomly allocated to the nodes but following iterative learning steps, the algorithm undergoes a training process that will result in a correct classification. During this process, the weighting of nodes change by repeated interaction with the items of the dataset in a way that captures the distribution of variability of the dataset. Thus, similar gene expression patterns map together in the network and, as far as possible, from the different patterns. At the end of the training process, the nodes of the SOM grid have clusters of related gene expression patterns assigned, and the trained nodes represent an average pattern of the cluster of data that map into it.

For example, profiling can be performed using Affymetrix GENECHIP® expression arrays and Affymetrix processing tools. Transcriptional analyses can be performed using Affymetrix U133 v2.0 gene arrays after globin reduction. RNA is prepared using standard protocols and hybridized to Affymetrix Human Genome U133 plus 2.0 arrays. Data can be processed using Bioconductor, a software, primarily based on R programming language for the analysis and comprehension of genomic data (Bioconductor.org). The data can be preprocessed using GCRMA package in Bioconductor, which normalizes at the probe level using the GC content of probes in normalization with RMA (robust multi-array).

Statistically significant differentially expressed genes between the control and test animal can be identified using SAM algorithm (Significance Analysis of Microarrays) with a false discovery rate of 5% and so forth.

Commercially available arrays include: ATLAS™ arrays from Clontech; GENECHIP® expression arrays from Affymetrix; and Agilent gene expression arrays. Array software and services are available from, e.g., Clontech; Affymetrix; and Agilent.

In Vivo Imaging

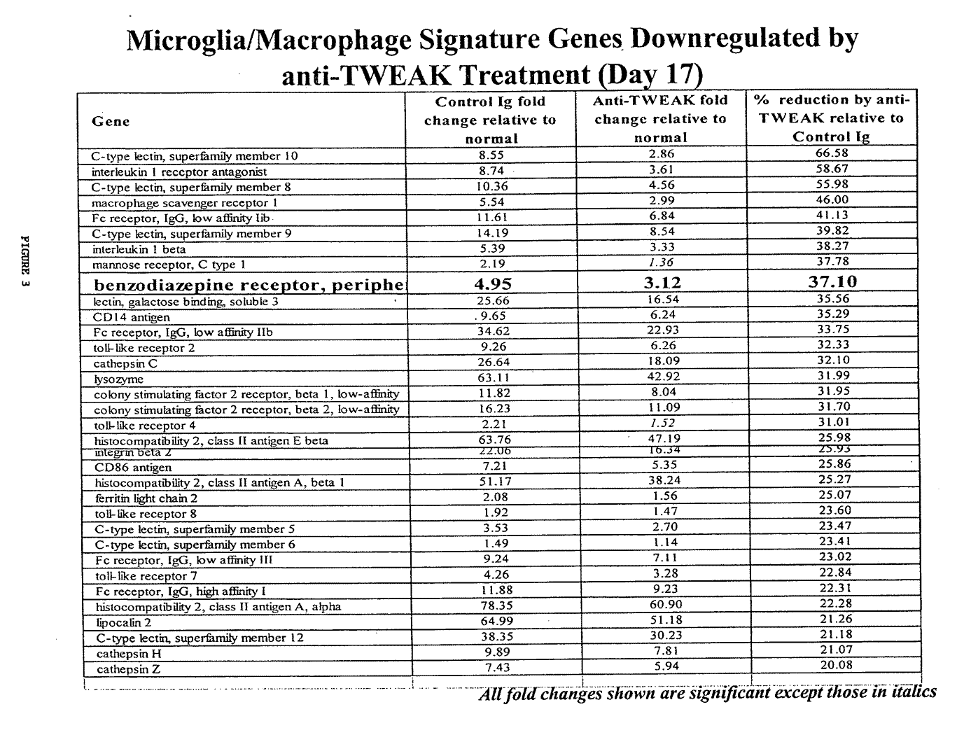

An agent (e.g., a ligand or antibody) that binds to PBR, or to another biomarker of MS described herein, provides a method for detecting the presence of a parameter associated with PBR (e.g., PBR nucleic acid or protein levels), or other biomarker, in vivo (e.g., in vivo imaging in a subject). The biomarker can be, for example, PBR or another gene listed in FIG. 3 (or protein product thereof), soluble ferritin (protein or nucleic acid, e.g., in cerebrospinal fluid), or soluble CD14 (protein or nucleic acid, e.g., in cerebrospinal fluid). The method can be used to evaluate the efficacy of anti-TWEAK/TWEAK receptor therapy in treating MS. For example, the method includes: (i) administering to a subject (and optionally a control subject) a PBR binding agent (e.g., an antibody or antigen binding fragment thereof, although such agents need not be blocking agents, or ligand), under conditions that allow interaction of the binding agent and PBR to occur; and (ii) detecting localization of the binding agent in the subject. The method can be used to detect the location of PBR expressing cells and levels of PBR in a subject or in a region, e.g., in the CNS, e.g., brain or spinal cord. A decrease, e.g., statistically significant decrease, in the amount of the complex in the subject relative to the reference, e.g., a control subject (e.g., untreated MS patient) or subject's baseline, can be a factor indicating that the anti-TWEAK/TWEAK-R therapy is affecting the progression or symptoms of MS. These methods can also be performed with another biomarker described herein.

The PBR (or other biomarker) binding agent can be used in in vivo (and can also be used in vitro) diagnostic methods and can be directly or indirectly labeled with a detectable substance to facilitate detection of the bound or unbound binding agent. Suitable detectable substances include various enzymes, prosthetic groups, fluorescent materials, luminescent materials and radioactive materials. In one embodiment, the PBR binding agent (e.g., protein) is coupled to a radioactive ion, e.g., indium (¹¹¹In), iodine (¹³¹I or ¹²⁵I), yttrium (⁹⁰Y), actinium (²²⁵Ac), bismuth (²¹²Bi or ²¹³Bi), sulfur (³⁵S), carbon (¹⁴C or ¹¹C), tritium (³H), rhodium (¹⁸⁸Rh), technicium (^{99m}Tc) or phosphorous (³²P or ³³P). In another embodiment, the PBR binding agent is labeled with an NMR contrast agent. In another embodiment, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short range radiation emitters, such as isotopes detectable by short range detector probes, can also be employed. The protein ligand can be labeled with such reagents using known techniques. For example, see Wensel and Meares (1983) Radioimmunoimaging and Radioimmunotherapy, Elsevier, New York for techniques relating to the radiolabeling of antibodies and Colcher et al. (1986) Meth. Enzymol. 121:802-816.

In one aspect, the invention features a method of imaging the central nervous system (CNS) or regions thereof (e.g., spinal cord or brain) in a subject who is at risk for MS or has been diagnosed as having MS, e.g., is suffering a relapse. The imaging methods can also be used as part of making a diagnosis of MS. The method includes: providing an agent that binds to PBR, e.g., an agent described herein, wherein the agent is physically associated to an imaging agent; administering the agent to a subject, e.g., with a risk for MS or who has been diagnosed as having MS, e.g., to detect PBR binding. For example, binding can be detected by NMR or PET imaging.

For example, a PBR binding agent (e.g., ligand or antibody) can be administered to a subject to detect PBR within the subject. For example, the antibody can be labeled, e.g., with an MRI detectable label or a radiolabel. The subject can be evaluated using a means for detecting the detectable label. For example, the subject can be scanned to evaluate localization of the antibody within the subject. For example, the subject is imaged, e.g., by NMR, PET, or other tomographic means. These methods can also be performed with another biomarker described herein.

The subject can be “imaged” in vivo using known techniques such as radionuclear scanning using e.g., a gamma camera or emission tomography. See e.g., A. R. Bradwell et al. “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated PET VI located at Brookhaven National Laboratory, can be used where the radiolabel emits positrons (e.g., ¹¹C, ¹⁸F, ¹⁵O, and ¹³N).

Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EPO 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents, paramagnetic agents (which primarily alter T1) and ferromagnetic or superparamagnetic agents (which primarily alter T2 response). Chelates (e.g., EDTA, DTPA and NTA chelates) can be used to attach (and reduce toxicity) of some paramagnetic substances (e.g., Fe³⁺, Mn²⁺, Gd³⁺). Other agents can be in the form of particles, e.g., less than 10 μm to about 10 nm in diameter). Particles can have ferromagnetic, anti-ferromagnetic or superparamagnetic properties. Particles can include, e.g., magnetite (Fe₃O₄), γ-Fe₂O₃, ferrites, and other magnetic mineral compounds of transition elements. Magnetic particles may include one or more magnetic crystals with and without nonmagnetic material. The nonmagnetic material can include synthetic or natural polymers (such as sepharose, dextran, dextrin, starch and the like).

The ligands and antibodies can also be labeled with an indicating group containing the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image PBR (or other biomarker) distribution.

Nucleic Acid and Protein Analysis

The nucleic acid or protein levels of PBR, or of another biomarker of MS described herein, can also be measured in a sample obtained from a subject, e.g., a subject with a risk for MS or who has been diagnosed as having MS, e.g., and is currently undergoing treatment for MS. The levels of PBR, or other biomarker described herein, can also be measured as part of making a diagnosis of MS. The levels can also be measured to evaluate the efficacy of anti-TWEAK/TWEAK-R therapy in treating MS.

Numerous methods for detecting PBR protein and nucleic acid, as well as proteins and nucleic acids for other biomarkers described herein (including those listed in FIG. 3, soluble ferritin, and soluble CD14), are available to the skilled artisan, including antibody-based methods for protein detection (e.g., Western blot, ELISA, or flow cytometry), and hybridization-based methods for nucleic acid detection (e.g., PCR (e.g., RT-PCR) or Northern blot).

Arrays are particularly useful molecular tools for characterizing a sample, e.g., a sample from a subject. For example, an array having capture probes for multiple genes, including probes for PBR and/or other biomarkers, or for multiple proteins, can be used in a method described herein. Altered expression of PBR (or other biomarker provided herein) nucleic acids and/or protein can be used to evaluate a sample, e.g., a sample from a subject, e.g., to evaluate the effects of anti-TWEAK/TWEAK-R therapy on MS, or to evaluate MS disease status or progression.

Arrays can have many addresses, e.g., locatable sites, on a substrate. The featured arrays can be configured in a variety of formats, non-limiting examples of which are described below. The substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular.

Arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145).

The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the gene. The Tₘ of the capture probe can be optimized by prudent selection of the complementarity region and length. Ideally, the Tₘ of all capture probes on the array is similar, e.g., within 20, 10, 5, 3, or 2° C. of one another.

The isolated nucleic acid is preferably mRNA that can be isolated by routine methods, e.g., including DNase treatment to remove genomic DNA and hybridization to an oligo-dT coupled solid substrate (e.g., as described in Current Protocols in Molecular Biology, John Wiley & Sons, N.Y). The substrate is washed, and the mRNA is eluted.

The isolated mRNA can be reversed transcribed and optionally amplified, e.g., by rtPCR, e.g., as described in (U.S. Pat. No. 4,683,202). The nucleic acid can be an amplification product, e.g., from PCR (U.S. Pat. Nos. 4,683,196 and 4,683,202); rolling circle amplification (“RCA,” U.S. Pat. No. 5,714,320), isothermal RNA amplification or NASBA (U.S. Pat. Nos. 5,130,238; 5,409,818; and 5,554,517), and strand displacement amplification (U.S. Pat. No. 5,455,166). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

The expression level of PBR or other biomarker can be determined using an agent, e.g., an antibody specific or a ligand specific for the polypeptide (e.g., using a Western blot, flow cytometry, or an ELISA assay). Moreover, the expression levels of multiple proteins, including PBR and the exemplary biomarkers provided herein (e.g., in FIG. 3, soluble ferritin, soluble CD14), or some combination of the biomarkers provided herein, can be rapidly determined in parallel using a polypeptide array having antibody capture probes for each of the polypeptides. Antibodies specific for a polypeptide can be generated by a method described herein (see “Antibody Generation”). Commercial antibodies and ligands (e.g., PK 11195) that specifically bind to PBR are also available. The expression level of a PBR and the exemplary biomarkers provided herein can be measured in a subject (e.g., in vivo imaging) or in vitro in a biological sample from a subject (e.g., blood, serum, plasma, cerebrospinal fluid, or urine).

A low-density (96 well format) protein array has been developed in which proteins are spotted onto a nitrocellulose membrane (Ge (2000) Nucleic Acids Res. 28, e3, I-VII). A high-density protein array (100,000 samples within 222×222 mm) used for antibody screening was formed by spotting proteins onto polyvinylidenc difluoride (PVDF) (Lueking et al. (1999) Anal. Biochein. 270:103-111). See also, e.g., Mendoza et al. (1999). Biotechniques 27:778-788; MacBeath and Schreiber (2000) Science 289:1760-1763; and De Wildt et al. (2000) Nature Biotech. 18:989-994. These art-known methods and other can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address.

The nucleic acid and polypeptide arrays of the invention can be used in a wide variety of applications. For example, the arrays can be used to analyze a subject sample. The sample is compared to data obtained previously, e.g., known clinical specimens or other subject samples. Further, the arrays can be used to characterize a cell culture sample, e.g., to determine a cellular state after varying a parameter, e.g., exposing the cell culture to an antigen, a transgene, or a test compound.

The expression data can be stored in a database, e.g., a relational database such as a SQL database (e.g., Oracle or Sybase database environments). The database can have multiple tables. For example, raw expression data can be stored in one table, wherein each column corresponds to a gene being assayed, e.g., an address or an array, and each row corresponds to a sample. A separate table can store identifiers and sample information, e.g., the batch number of the array used, date, and other quality control information.

Expression profiles obtained from gene expression analysis on an array can be used to compare samples and/or cells in a variety of states as described in Golub et al. ((1999) Science 286:531). In one embodiment, expression (e.g., mRNA expression or protein expression) information for a gene encoding PBR and/or a gene encoding a exemplary biomarker provided herein are evaluated, e.g., by comparison a reference value, e.g., a reference value. Reference values can be obtained from a control, e.g., a reference subject. Reference values can also be obtained from statistical analysis, e.g., to provide a reference value for a cohort of subject, e.g., age and gender matched subject, e.g., normal subjects or subject who have rheumatoid arthritis or other disorder described herein. Statistical similarity to a particular reference (e.g., to a reference for a risk-associated cohort) or a normal cohort can be used to provide an assessment (e.g., an indication of risk of inflammatory disorder) to a subject, e.g., a subject who has not been diagnosed with a disorder described herein.

Subjects suitable for treatment can also be evaluated for expression and/or activity of PBR and/or other biomarker. Subjects can be identified as suitable for treatment (e.g., with an anti-TWEAK antibody), if the expression and/or activity for PBR and/or the other biomarker is elevated relative to a reference, e.g., reference value, e.g., a reference value associated with normal.

Subjects who are being administered an agent described herein or other treatment for MS can be evaluated as described for expression of PBR and/or other biomarker described herein. The subject can be evaluated at multiple times. e.g., at multiple times during a course of therapy, e.g., during a therapeutic regimen. Treatment of the subject can be modified depending on how the subject is responding to the therapy. For example, a reduction in PBR expression or a reduction in the expression or activity of genes induced by MS can be indicative of responsiveness to a given therapy, e.g., anti-TWEAK antibody therapy.

Particular effects mediated by an agent may show a difference (e.g., relative to an untreated subject, control subject, subject's baseline before commencing treatment, or other reference) that is statistically significant (e.g., P value<0.05 or 0.02). Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02.

Methods of Evaluating Genetic Material

There are numerous methods for evaluating genetic material to provide genetic information. These methods can be used to evaluate a genetic locus that includes a gene encoding PBR or a gene encoding a biomarker described herein. The methods can be used to evaluate one or more nucleotides, e.g., a coding or non-coding region of the gene, e.g., in a regulatory region (e.g., a promoter, a region encoding an untranslated region or intron, and so forth).

Nucleic acid samples can analyzed using biophysical techniques (e.g., hybridization, electrophoresis, and so forth), sequencing, enzyme-based techniques, and combinations-thereof. For example, hybridization of sample nucleic acids to nucleic acid microarrays can be used to evaluate sequences in an mRNA population and to evaluate genetic polymorphisms. Other hybridization based techniques include sequence specific primer binding (e.g., PCR or LCR); Southern analysis of DNA, e.g., genomic DNA; Northern analysis of RNA, e.g., mRNA; fluorescent probe based techniques (see, e.g., Beaudet etal. (2001) Genome Res. 11(4):600-8); and allele specific amplification. Enzymatic techniques include restriction enzyme digestion; sequencing; and single base extension (SBE). These and other techniques are well known to those skilled in the art.

Electrophoretic techniques include capillary electrophoresis and Single-Strand Conformation Polymorphism (SSCP) detection (see, e.g., Myers et al. (1985) Nature 313:495-498 and Ganguly (2002) Hum Mutat. 19(4):334-342). Other biophysical methods include denaturing high pressure liquid chromatography (DHPLC).

In one embodiment, allele specific amplification technology that depends on selective PCR amplification may be used to obtain genetic information. Oligonucleotides used as primers for specific amplification may carry the mutation of interest in the center of the molecule (so that amplification depends on differential hybridization) (Gibbs et al. (1989) Nucl. Acids Res. 17:2437-2448) or at the extreme 3′ end of one primer where, under appropriate conditions, mismatch can prevent, or reduce polymerase extension (Prossner (1993) Tibtech 11:238). In addition, it is possible to introduce a restriction site in the region of the mutation to create cleavage-based detection (Gasparini et al. (1992) Mol. Cell Probes 6:1). In another embodiment, amplification can be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Enzymatic methods for detecting sequences include amplification based-methods such as the polymerase chain reaction (PCR; Saiki, et al. (1985) Science 230:1350-1354) and ligase chain reaction (LCR; Wu. et al. (1989) Genomics 4:560-569; Barringer et al. (1990), Gene 1989:117-122; F. Barany (1991) Proc. Natl. Acad. Sci. USA 1988:189-193); transcription-based methods utilize RNA synthesis by RNA polymerases to amplify nucleic acid (U.S. Pat. Nos. 6,066,457; 6,132,997; and 5,716,785; Sarkar et al., (1989) Science 244:331-34; Stofler et al., (1988) Science 239:491); NASBA (U.S. Pat. Nos. 5,130,238; 5,409,818; and 5,554,517); rolling circle amplification (RCA; U.S. Pat. Nos. 5,854,033 and 6,143,495) and strand displacement amplification (SDA; U.S. Pat. Nos. 5,455,166 and 5,624,825). Amplification methods can be used in combination with other techniques.

Other enzymatic techniques include sequencing using polymerases, e.g., DNA polymerases and variations thereof such as single base extension technology. See, e.g., U.S. Pat. Nos. 6,294,336; 6,013,431; and 5,952,174.

Fluorescence based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (e.g., “color”) of the fluorescent dye that is incorporated.

Hybridization to microarrays can also be used to detect polymorphisms, including SNPs. For example, a set of different oligonucleotides, with the polymorphic nucleotide at varying positions with the oligonucleotides can be positioned on a nucleic acid array. The extent of hybridization as a function of position and hybridization to oligonucleotides specific for the other allele can be used to determine whether a particular polymorphism is present. See, e.g., U.S. Pat. No. 6,066,454.

In one implementation, hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other.

It is also possible to directly sequence the nucleic acid for a particular genetic locus, e.g., by amplification and sequencing, or amplification, cloning and sequence. High throughput automated (e.g., capillary or microchip based) sequencing apparati can be used. In still other embodiments, the sequence of a protein of interest is analyzed to infer its genetic sequence. Methods of analyzing a protein sequence include protein sequencing, mass spectroscopy, sequence/epitope specific immunoglobulins, and protease digestion.

Any combination of the above methods can also be used.

TWEAK/TWEAK Receptor Blocking Agents

An exemplary sequence of a human TWEAK protein is as follows:

(SEQ ID NO: 1)
MAARRSQRRR GRRGEPGTAL LVPLALGLGL ALACLGLLLA
VVSLGSRASL SAQEPAQEEL VAEEDQDPSE LNPQTEESQD
PAPFLNRLVR PRRSAPKGRK TRARRAIAAH YEVHPRPGQD
GAQAGVDGTV SGWEEARINS SSPLRYNRQI GEFIVTRAGL
YYLYCQVHFD EGKAVYLKLD LLVDGVLALR CLEEFSATAA
SSLGPQLRLC QVSGLLALRP GSSLRIRTLP WAHLKAAPFL
TYFGLFQVH

An exemplary sequence of a human TWEAK receptor protein is as follows:

(SEQ ID NO: 2)
MARGSLRRLL RLLVLGLWLA LLRSVAGEQA PGTAPCSRGS
SWSADLDKCM DCASCRARPH SDFCLGCAAA PPAPFRLLWP
ILGGALSLTF VLGLLSGFLV WRRCRRREKF TTPIEETGGE
GCPAVALIQ

A variety of agents can be used as a TWEAK/TWEAK-R blocking agent to treat MS. The agent may be any type of compound (e.g., small organic or inorganic molecule, nucleic acid, protein, or peptide mimetic) that can be administered to a subject. In one embodiment, the blocking agent is a biologic, e.g., a protein having a molecular weight of between 5-300 kDa. For example, a TWEAK/TWEAK-R blocking agent may inhibit binding of TWEAK to a TWEAK receptor or may prevent TWEAK-mediated NF-KB activation. A typical TWEAK/TWEAK-R blocking agent can bind to TWEAK or a TWEAK receptor, e.g., Fn14. A TWEAK/TWEAK-R blocking agent that binds to TWEAK may alter the conformation of TWEAK or a TWEAK receptor, block the binding site on TWEAK or a TWEAK receptor, or otherwise decrease the affinity of TWEAK for a TWEAK receptor or prevent the interaction between TWEAK and a TWEAK receptor. A TWEAK/TWEAK-R blocking agent (e.g., an antibody) may bind to TWEAK or to a TWEAK receptor with a K_{d} of less than 10⁻⁶, 10⁻⁷, 10⁻⁸, 10⁻⁹, or 10⁻¹⁰ M. In one embodiment, the blocking agent binds to TWEAK with an affinity at least 5, 10, 20, 50, 100, 200, 500, or 1000 better than its affinity for TNF or another TNF superfamily member (other than TWEAK). In one embodiment, the blocking agent binds to the TWEAK receptor with an affinity at least 5, 10, 20, 50, 100, 200, 500, or 1000-fold better than its affinity for the TNF receptor or a receptor for another TNF superfamily member. A preferred TWEAK/TWEAK-R blocking agent specifically binds TWEAK or TWEAK-R, such as a TWEAK or TWEAK-R specific antibody.

In some embodiments, the TWEAKJTWEAK R blocking agent is an engineered binding protein. Briefly, a library that contains a scaffold protein framework (typically of defined length), in which random or selected positions are varied, is prepared. Positions in the scaffold can be varied, e.g., by use of degenerate codons. The scaffold library (e.g., phage display library) is screened for proteins that bind a target of interest (e.g., TWEAK or TWEAK-R). Proteins binding with the desired affinity or other desired characteristic are selected. Numerous scaffolds are available, e.g., fibronectin-, affibody- (Protein A based), lipocalin-, DARPin- (designed ankyrin-repeat protein), Src homoloyg domain 2-, Src homology domain 3-, β-lactamase-, small disulfide-bonded-, and protease inhibitor-based scaffolds. For a review of scaffold technology, see Binz et al., (2005) Nat. Biotech. 23:1257-1268.

Exemplary TWEAK protein molecules include human TWEAK (e.g., AAC51923, shown as SEQ ID NO:1)), mouse TWEAK (e.g., NP_{—}035744.1), rat TWEAK (e.g., XP_{—}340827.1), and Pan troglodytes TWEAK (e.g., XP_{—}511964.1). Also included arc proteins that include an amino acid sequence at least 90, 92, 95, 97, 98, 99% identical and completely identical to the mature processed region of the aforementioned TWEAK proteins (e.g., an amino acid sequence at least 90, 92, 95, 97, 98, 99% identical or completely identical to amino acids X₁-249 of SEQ ID NO:1, where amino acid X₁ is selected from the group of residues 75-115 of SEQ ID NO:1, e.g., X₁ is residue Arg 93 of SEQ ID NO:1) and proteins encoded by a nucleic acid that hybridizes under high stringency conditions to a human, mouse, rat, or Pan troglodytes gene encoding a naturally occurring TWEAK protein. Preferably, a TWEAK protein, in its processed mature form, is capable of providing at least one TWEAK activity, e.g., ability to activate Fn14.

Exemplary Fn14 protein molecules include human Fn14 (e.g., NP_{—}057723.1, shown as SEQ ID NO:2), mouse Fn14 (e.g., NP_{—}038777.1), and rat Fn14 (e.g., NP_{—}851600.1) as well as soluble proteins that include an amino acid sequence at least 90, 92, 95, 97, 98, 99% identical to the extracellular domain of Fn14 (and TWEAK-binding fragments thereof) and proteins encoded by a nucleic acid that hybridizes under high stringency conditions to a human, mouse, rat, or Pan troglodytes gene encoding a naturally occurring Fn14 protein. Preferably, a Fn14 protein useful in the methods described herein is a soluble Fn14 (lacking a transmembrane domain) that includes a region that binds to a TWEAK protein, e.g., an amino acid sequence at least 90, 92, 95, 97, 98, or 99% identical, or completely identical, to amino acids 28-X₁ of SEQ ID NO:2, where amino acid X₁ is selected from the group of residues 68 to 80 of SEQ ID NO:2.

Calculations of “homology” or “sequence identity” between two sequences (the terms arc used interchangeably herein) arc performed as follows. The sequences arc aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. High stringency hybridization conditions include hybridization in 6×SSC at about 45° C., followed by one or more washes in 0.2×SSC, 0.1% SDS at 65° C., or substantially similar conditions.

Exemplary TWEAK/TWEAK-R blocking agents include antibodies that bind to TWEAK or TWEAK-R and soluble forms of the TWEAK-R that compete with cell surface TWEAK-R for binding to TWEAK. An example of a soluble form of the TWEAK-R is an Fc fusion protein that includes at least a portion of the extracellular domain of TWEAK-R (e.g., a soluble TWEAK-binding fragment of TWEAK-R), referred to as TWEAK-R-Fc. Other soluble forms of TWEAK-R, e.g., forms that do not include an Fc domain, can also be used. Antibody blocking agents are further discussed below. Other types of blocking agents, e.g., small molecules, nucleic acid or nucleic acid-based aptamers, and peptides, can be isolated by screening, e.g., as described in Jhaveri et al. Nat. Biotechnol. 18:1293 and U.S. Pat. No. 5,223,409. Exemplary assays for determining if an agent binds to TWEAK or TWEAK-R and for determining if an agent modulates a TWEAK/TWEAK-R interaction are described, e.g., in US 2004-0033225.

An exemplary soluble form of the TWEAK-R protein includes a region of the TWEAK-R protein that binds to TWEAK, e.g., about amino acids 32-75, 31-75, 31-78, or 28-79 of SEQ ID NO:2. This region can be physically associated, e.g., fused to another amino acid sequence, e.g., an Fc domain, at its N- or C-terminus. The region from TWEAK-R can be spaced by a linker from the heterologous amino acid sequence. U.S. Pat. No. 6,824,773 describes an exemplary TWEAK-R fusion protein.

Antibodies

Exemplary TWEAK/TWEAK-R blocking agents include antibodies that bind to TWEAK and/or TWEAK-R. In one embodiment, the antibody inhibits the interaction between TWEAK and a TWEAK-R, e.g., by physically blocking the interaction, decreasing the affinity of TWEAK and/or TWEAK-R for its counterpart, disrupting or destabilizing TWEAK complexes, sequestering TWEAK or a TWEAK-R, or targeting TWEAK or TWEAK-R for degradation. In one embodiment, the antibody can bind to TWEAK or TWEAK-R at one or more amino acid residues that participate in the TWEAK/TWEAK-R binding interface. Such amino acid residues can be identified, e.g., by alanine scanning. In another embodiment, the antibody can bind to residues that do not participate in the TWEAK/TWEAK-R binding. For example, the antibody can alter a conformation of TWEAK or TWEAK-R and thereby reduce binding affinity, or the antibody may sterically hinder TWEAK/TWEAK-R binding. In one embodiment, the antibody can prevent activation of a TWEAK/TWEAK-R mediated event or activity (e.g., NF-κB activation).

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (II) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The term also encompasses bispecific antibodies and bispecific antibody fragments (e.g., diabodies). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, US Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally-occurring variable domain. For example, the sequence may omit one, two, or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with TWEAK or a TWEAK receptor.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. FIC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

Exemplary anti-TWEAK antibodies, pharmaceutical compositions containing such antibodies, modes of administering such antibodies and compositions, and devices and kits containing such antibodies and compositions, are described in International Application PCT/US2006/019706, filed on May 25, 2006.

The antibodies can be conjugated to a moiety, e.g., can be conjugated to poly(ethylene glycol) (e.g., PEGylated), e.g., to reduce the immunogenicity and/or increase the circulating half-lives of antibodies.

Antibody Generation

Antibodies that bind to TWEAK or a TWEAK-R can be generated by a variety of means, including immunization, e.g., using an animal, or in vitro methods such as phage display. All or part of TWEAK or a TWEAK receptor can be used as an immunogen or as a target for selection. For example, TWEAK or a fragment thereof, TWEAK-R or a fragment thereof, can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human ig loci. Using the hybridoma technology, antigen-specific monoclonal antibodies derived from the genes with the desired specificity may be produced and selected. See, e.g., XENOMOUSE™, Green et al. (1994) Nat. Gen. 7:13-21; US 2003-0070185; U.S. Pat. No. 5,789,650; and WO 96/34096.

Non-human antibodies to TWEAK or a TWEAK receptor can also be produced, e.g., in a rodent. The non-human antibody can be humanized, e.g., as described in EP 239 400; U.S. Pat. Nos. 6,602,503; 5,693,761; and 6,407,213, deimmunized, or otherwise modified to make it effectively human.

EP 239 400 (Winter et al.) describes altering antibodies by substitution (within a given variable region) of their complementarity determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody. Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; and 5,530,101; Tempest et al. (1991) Biotechnology 9:266-271 and U.S. Pat. No. 6,407,213.

Fully human monoclonal antibodies that bind to TWEAK or a TWEAK receptor can be produced, e.g., using in vitro-primed human splenocytes, as described by Boerner et al. (1991) J. Immunol. 147:86-95. They may be prepared by repertoire cloning as described by Persson et al. (1991) Proc. Nat. Acad. Sci. USA 88:2432-2436 or by Huang and Stollar (1991) J. Immunol. Methods 141:227-236; also U.S. Pat. No. 5,798,230. Large nonimmunized human phage display libraries may also be used to isolate high affinity antibodies that can be developed as human therapeutics using standard phage technology (see, e.g., Hoogenboom et al. (1998) Immunotechnology 4:1-20; Hoogenboom et al. (2000) Immunol Today 2:371-378; and US 2003-0232333).

Antibody and Protein Production

Antibodies and other proteins described herein can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-35), Hanseula, or Saccharomyces.

Antibodies, particularly full length antibodies, e.g., IgG's, can be produced in mammalian cells. Exemplary mammalian host cells for recombinant expression include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NSO myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies (and Fe fusions) may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some proteins that include an Fc domain, the antibody/protein production system may be designed to synthesize antibodies or other proteins in which the Fc region is glycosylated. For example, the Fc domain of IgG molecules is glycosylated at asparagine 297 in the CH2 domain. The Fc domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the protein of interest, e.g., an antibody or Fc fusion protein. The protein can be purified from the milk, or for some applications, used directly.

Methods described in the context of antibodies can be adapted to other proteins, e.g., Fc fusions and soluble receptor fragments.

Nucleic Acid Blocking Agents

In certain implementations, nucleic acid blocking agents are used to decrease expression of an endogenous gene encoding TWEAK or a TWEAK-R, e.g., Fn14. In one embodiment, the nucleic acid antagonist is an siRNA that targets mRNA encoding TWEAK or a TWEAK-R. Other types of antagonistic nucleic acids can also be used, e.g., a dsRNA, a ribozyme, a triple-helix former, or an antisense nucleic acid.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature 411:494-498; Yang et al. (2002) Proc. Natl. Acad. Sci. USA 99:9942-9947, U.S. 2003-0166282; 2003-0143204; 2004-0038278; and 2003-0224432.

Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA (e.g., an mRNA encoding TWEAK or TWEAK-R) can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding TWEAK or TWEAK-R. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂) alkylaminocytosines and N⁴,N⁴—(C₁-C₁₂) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂) alkylaminopurines and N⁶,N⁶—(C₁-C₁₂) dialkylaminopurines, including N⁶ -rnethylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. Nos. 4,987,071;. 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-7309; Haseloff and Gerlach (1988) Nature 334:585-591; Helene, C. (1991) Anticancer Drug Des. 6:569-584; Helene (1992) Ann. N.Y. Acad. Sci. 660:27-36; and Maher (1992) Bioassays 14:807-815.

The nucleic acids described herein, e.g., an anti-sense nucleic acid described herein, can be incorporated into a gene construct to be used as a part of a gene therapy protocol to deliver nucleic acids that can be used to express and produce agents, e.g., anti-sense nucleic acids within cells. Expression constructs of such components may be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g. a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which arc known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Inununol. 150:4104-4115; U.S. Pat. No. 4,868,116; U.S. Pat. No. 4,980,286; PCT Application WO 89/07136; PCT Application. WO 89/02468; PCT Application WO 89/05345; and PCT Application WO 92/07573).

Another viral gene delivery system utilizes adenovirus-derived vectors. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are known to those skilled in the art.

Yet another viral vector system useful for delivery of the subject gene is the adeno-associated virus (AAV). See, for example, Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973).

Artificial Transcription Factors

Artificial transcription factors can also be used to regulate expression of TWEAK and/or TWEAK-R. The artificial transcription factor can be designed or selected from a library, e.g., for ability to bind to a sequence in an endogenous gene encoding TWEAK or TWEAK-R, e.g., in a regulatory region, e.g., the promoter. For example, the artificial transcription factor can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19:656; and Wu et al. (1995) Proc. Natl. Acad. Sci. USA 92:344 for, among other things, methods for creating libraries of varied zinc finger domains.

Optionally, an artificial transcription factor can be fused to a transcriptional regulatory domain, e.g., an activation domain to activate transcription or a repression domain to repress transcription. In particular, repression domains can be used to decrease expression of endogenous genes encoding TWEAK or TWEAK-R. The artificial transcription factor can itself be encoded by a heterologous nucleic acid that is delivered to a cell or the protein itself can be delivered to a cell (see, e.g., U.S. Pat. No. 6,534,261). The heterologous nucleic acid that includes a sequence encoding the artificial transcription factor can be operably linked to an inducible promoter, e.g., to enable fine control of the level of the artificial transcription factor in the cell, e.g., a neuronal or glial cells, e.g., at or near a site of neuronal or other injury in the brain or spinal cord or at the site of neurodegeneration caused by a neurological disorder.

Kits

A nucleic acid probe that detects PBR, or another biomarker described herein, can be used as a component of a kit or as a reagent, e.g., a diagnostic kit or diagnostic reagent. For example, a nucleic acid (or its complement) (e.g., an oligonucleotide, e.g., probe) corresponding to one or more of the genes described herein (or one or more signature sets described herein) can be a member of a nucleic acid array against which a sample (e.g., from a subject, e.g., a subject who has MS) is hybridized to determine the level of gene expression. For example, a probe for PBR or a biomarker described herein can be present on an array for a TAQMAN® gene expression assay (Applied Biosystems) (e.g., a custom TAQMAN® assay), e.g., for use in a 384-well plate format, e.g., using standard protocols. The diagnostic evaluation of a subject's sample (e.g., blood) can be performed, e.g., in a doctor's office, hospital laboratory, or contract laboratory.

The nucleic acid can contain the full length gene transcript (or its complement), or a fragment of the transcript (or its complement) (e.g., an oligonucleotide, e.g., probe) that allows for it to specifically bind to the nucleic acid complement (or the nucleic acid) in the sample under selected hybridization conditions. The level can then be compared to a control or reference value. The control or reference value can be part of the kit, or alternatively, the kit can contain the world wide web address on which reference information is located. Alternatively, nucleic acid (or its complement) corresponding to one or more of the genes described herein can be provided as a reagent (e.g., diagnostic reagent) that can be used to detect the presence and level of a gene transcript described herein. For example, the nucleic acid (or its complement) can be labeled with a detectable label and hybridized with nucleic acid from a sample. The level of hybridization can then be compared to a reference value. The reference value can be provided with the reagent, or alternatively, the reagent can contain a world wide web address for a site on which reference information is located.

Likewise, the polypeptide corresponding to a gene (e.g., a biomarker) described herein can be used as a reagent or as a component of a kit. The polypeptide can be the full length polypeptide or a fragment thereof that allows for it to specifically bind to an antibody or a ligand (e.g., receptor ligand or binding partner or fragment thereof) that is specific for the protein from which the fragment derives, or otherwise allow specific identification of the protein.

In another embodiment, antibodies (including intact and/or full length immunoglobulins of types IgA, IgG (e.g., IgG1, IgG2, IgG3, IgG4), IgE, IgD, IgM (as well as subtypes thereof) and antibody fragments, e.g., single chain antibodies, Fab fragments, F(ab′)2 fragments, Fd fragments, 1⁷v fragments, and dAb fragments) specific for one or more polypeptides encoded by gene transcripts of a biomarker described herein (e.g., anti-PBR antibody) can be a reagent or component of a kit for the detection of the polypeptide (e.g., PBR). For example, a sample (e.g., blood) can be contacted with the antibody (e.g., antibody that is labeled with a detectable label) under conditions that allow for binding of the antibody to its antigen and the presence and/or amount of binding are then detected (e.g., by ELISA).

In another embodiment, ligands (e.g., PK 11195) specific for one or more polypeptides encoded by gene transcripts of a biomarker described herein (e.g., PBR) can be a reagent or component of a kit for the detection of the polypeptide. For example, a sample (e.g., blood) can be contacted with the ligand (e.g., ligand that is labeled with a detectable label) under conditions that allow for binding of the ligand to the biomarker and the presence and/or amount of binding are then detected (e.g., by ELISA).

Any of the kits can optionally include instructions for its use (e.g., how to use the kit to predict a treatment outcome or to select a treatment regimen, etc.) or can contain a world wide web address to a link where instructions are provided. The reagents may also be supplied with instructions for their use (e.g., how to use the reagents to predict a treatment outcome or to select a treatment regimen, etc.) or a world wide web address to a link where instructions are provided.

The kits can be used, for example, to diagnose MS, predict the treatment outcome of a subject who has MS (e.g., if the subject is administered a particular therapy), select a treatment regimen (e.g., a monotherapy or combination.therapy), select dosages of a given treatment, monitor the course of a treatment for MS (e.g., to determine is the treatment needs to be altered), and/or select the duration of a treatment regimen.

Other Applications

The description herein provides examples of identification and analysis of biomarkers that are upregulated in MS, and for example, use of these biomarkers to evaluate and monitor efficacy of MS therapy (e.g., prevention or limitation of the upregulation of a biomarker described herein after administering a therapy suggests the therapy is effective in treating MS). The markers can further be used, for example, for testing potential MS therapies, for MS diagnosis, and even for delay or prevention of MS.

As will be appreciated, using the disclosure provided herein, a skilled practitioner will be able to apply like methods to identify biomarkers that are downregulated in MS. An MS therapy (e.g., a TWEAK-TWEAK-R blocking agent) can limit or prevent the MS-associated downregulation of the gene. In some embodiments, the therapy can contribute to amelioration of the symptoms and/or progression of MS. Likewise, in some embodiments, the therapy can upregulate expression of a gene that is normally downregulated during the course of MS, e.g., in a subject that has been identified as being at risk for developing MS. These biomarkers can be used in methods described herein, e.g., for evaluating and monitoring efficacy of MS therapy, for testing potential MS therapies, for MS diagnosis, and even for delay or prevention of MS.

### Examples

Anti-TWEAK Blocking Monoclonal Antibodies Reduce Clinical Severity in MOG-Induced EAE

EAE was induced by immunizing C57BL6 mice with MOG 35-55 peptide in the presence of adjuvant.

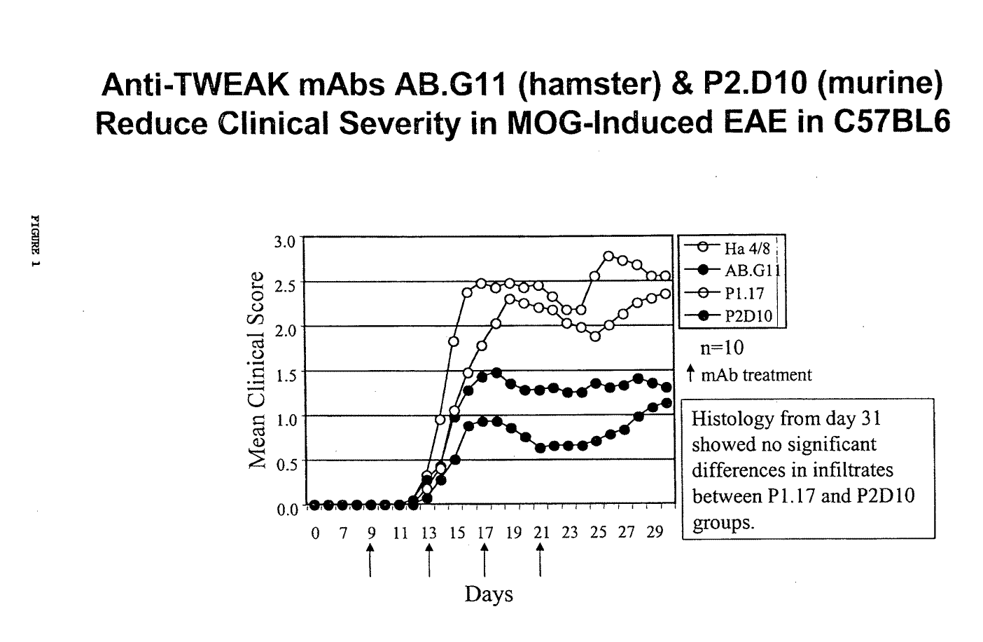

The EAE mice were treated with hamster anti-TWEAK mAb (AB.G11), isotype-matched control hamster IgG (HA4/8), murine anti-TWEAK mAb (P2D10), or isotype-matched control murine IgG (P1.17) at 9, 13, 17, and 21 days post immunization. 200 μg of antibody were administered per injection. The clinical course of disease was assessed on a clinical scale of 0 to 6 with the following criteria: 0: no detectable sign of EAE; 1: weakness of the tail; 2: definite tail paralysis and hind limb weakness; 3: partial paralysis of hind limbs; 4: complete paralysis of hind limbs; 5: complete paralysis of hind limbs with incontinence and partial or complete paralysis of forelimbs; 6: dead, and the mean clinical score calculated for each treatment over a time course. The results are shown in FIG. 1. As indicated in the figure, the mice treated with AB.G11 or P2D10 had lower mean clinical scores than the mice treated either control antibody. Histology performed at day 31 after disease induction showed no significant differences in infiltrates between P1.17 and P2D10 groups (data not shown).

Gene Profiling

EAE was induced by immunizing mice with MOG 35-55 peptide in presence of the adjuvant.

Mice were treated with anti-TWEAK mAb (AB.G11) or control IgG (HA4/8) at 9 and 13 days post immunization. Four days after the first sign of disease/disease onset (day 17), spinal cords were harvested and. RNA samples were prepared. RNA samples were used to obtain gene transcript profiles in spinal cords.

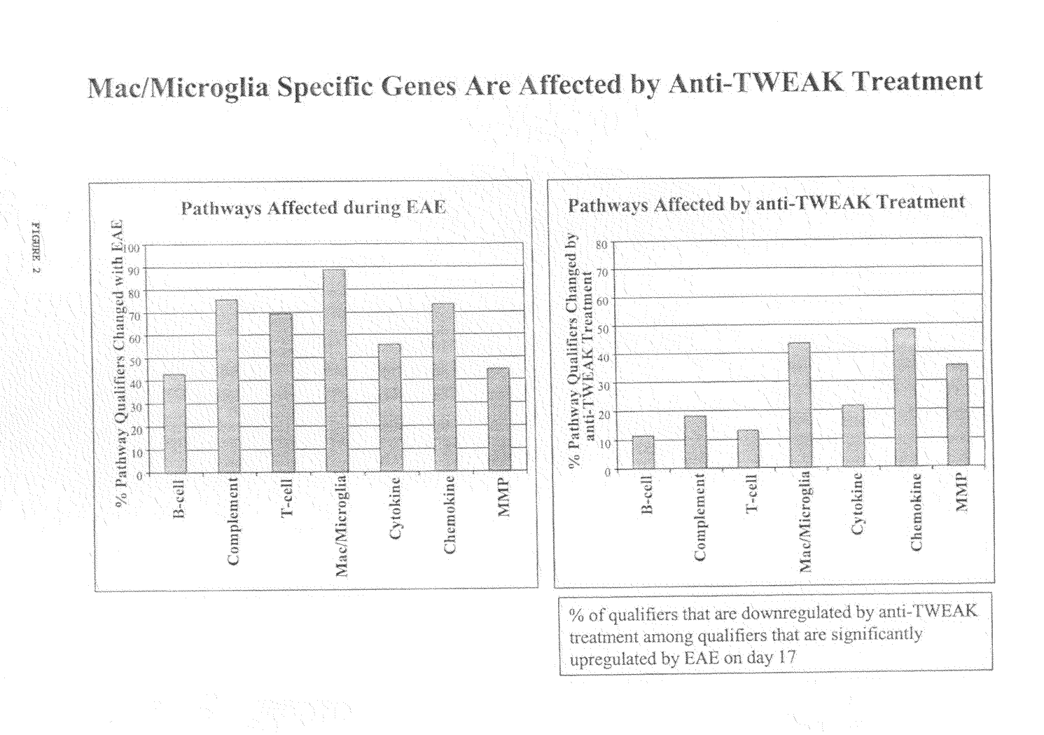

Gene transcript profile data from anti-TWEAK mAb treated and HA4/8 treated mice were analyzed by fold changes compared to normal mice. Analysis showed that macrophage/microglia associated genes are more specifically targeted by anti-TWEAK treatment. FIG. 2A shows that genes in the B cell, complement, T cell, cytokine, chemokine, and MMP pathways are also upregulated in EAE, and FIG. 2B shows that this upregulation is limited by treatment with anti-TWEAK therapy.

The table in FIG. 3 lists microglia/macrophage signature genes that are induced by EAE induction and whose upregulation is limited by anti-TWEAK mAb treatment of the EAE animals. The second column, labeled “Control Ig fold change relative to normal,” shows the fold increase in expression in EAE mice after treatment with the control Ha 4/8 antibody. The third column, labeled “Anti-TWEAK fold change relative to normal,” shows the fold increase in expression in EAE mice after treatment with the anti-TWEAK antibody. The last column, labeled “% reduction by anti-TWEAK relative to Control Ig,” shows the percent decrease in gene expression from the second to the third columns. One of the genes upregulated in EAE, whose upregulation is limited by anti-TWEAK treatment of EAE, is the peripheral benzodiazepine receptor (PBR).

Binding of PBR Ligand in Spinal Cord Extracts and Membranes

Spinal cord extracts from normal, control mAb treated- and anti-TWEAK mAb treated-EAE animals are tested for PBR ligand binding. The effect of anti-TWEAK treatment in limiting the upregulation of PBR is quantified by measuring PBR ligand binding in spinal cord protein extracts. The extracts are prepared for the following groups of mice (5 mice per group): normal mice, anti-TWEAK mAb (AB.G11) treated EAE mice, and control antibody (Ha 4/8) treated EAE mice. Membrane binding studies on whole cord are processed as one sample without any anatomical separation.

Frozen spinal cord tissue is homogenized in ice cold phosphate buffered saline (PBS) and centrifuged at 50,000 g for 10 minutes at 4° C. [³H]-PK11195 binding is assayed in an incubation volume of 1 ml, consisting of 0.5 ml membrane suspension and 0.5 ml [³H]-PK11195 (NEN, Boston, Mass., 85 Ci/mmol specific activity, at a final concentration of 6 nM, in the absence or presence of 1 μM RO-054864 (20 μl) to determine non specific binding. The assay mixtures are incubated at 4° C. for 120 minutes. Incubation is stopped by rapid filtration in vacuo through GF/B fiber filters which are washed with 12 ml of cold PBS and counted in 4 ml of Filter Count (Packard, Meriden, Conn.) in a liquid scintillation counter with a counting efficiency of 60%. See, e.g., Agnello et al., (2000) J Neuroimmunol. 109:105-11.

### TWEAK Levels are Upregulated in the Serum of MS Patients

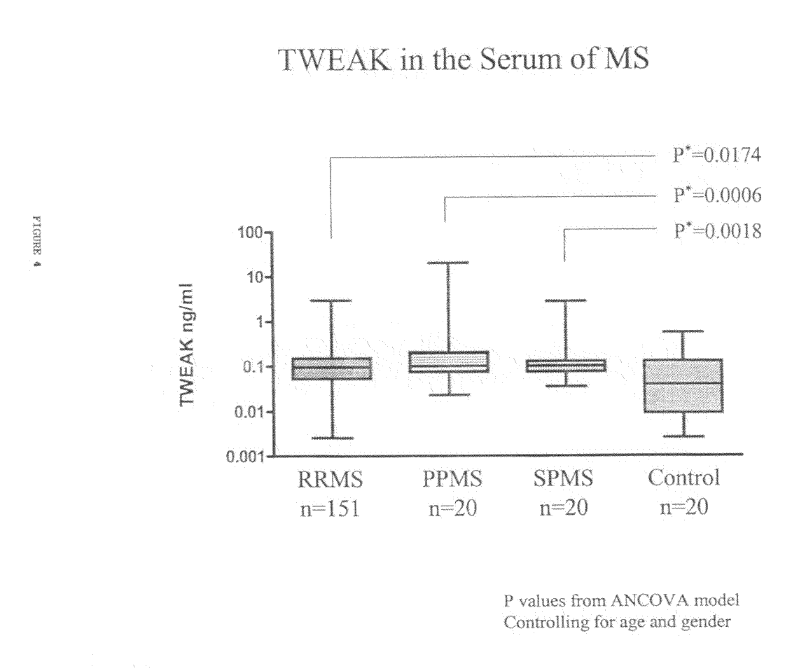

TWEAK levels were measured in sera of a control population and different MS populations: relapsing-remitting (RRMS), primary progressive (PPMS), and secondary progressive (SPMS) using ELISA. MS patients of all three populations showed elevated levels of TWEAK in sera compared to the control (FIG. 4). The P values shown in the figure were calculated by the ANCOVA model and controlling for age and gender.

Binding of PBR Ligand in Spinal Cord Sections by Autoradiography

PBR ligand binding is measured by autoradiography to localize and quantify sites and intensity of PBR ligand binding in the brain and spinal cord. Spinal cord and brain sections from EAE animals treated with anti-TWEAK or control antibodies are subjected to binding to radiolabeled PBR ligands, such as ³H-(R)-PK 11195. Location and intensity of PBR ligand binding are measured by autoradiography.

Biomarkers of Anti-TWEAK Pathway Therapy for Multiple Sclerosis

A gene product (nucleic acid or protein) identified as being upregulated in MS and whose upregulation is limited or prevented by treatment targeting the TWEAK signaling pathway is used as a biomarker to measure the effectiveness of the therapy in a subject that has MS. For example, PBR is used as a biomarker of anti-TWEAK therapy for MS.

An agent that binds to PBR protein, such as a PBR ligand or anti-PBR antibody, is labeled with a detectable label, administered to a subject, and the levels of binding to PBR are measured. For example, a carbon-11-labeled, R-enantiomer form of PK11195 ([¹¹C](R)-PK11195) is administered to a subject and its binding to PBR in the brain and spinal cord measured by PET scan. A first measurement of PBR binding is taken for the subject before commencing anti-TWEAK therapy to establish a baseline value. A second measurement is taken after anti-TWEAK therapy has commenced. A decrease in PBR levels, where levels are measured by the amount of PBR binding by the PBR-binding agent, after the commencement of anti-TWEAK therapy, as compared to the levels before therapy commenced, correlates with and is indicative of effective anti-TWEAK therapy.

Likewise, levels of another gene listed in FIG. 3, soluble ferritin, and/or soluble CD14 are measured in a similar fashion during the course of treatment for MS with anti-TWEAK antibody, or with another agent that targets the TWEAK signaling pathway.

Testing the Efficacy and Dosage of an MS Therapy

The efficacy of a potential MS therapy is tested in EAE mice. EAE is induced in the mice by MOG immunization. The potential therapy is administered 9 and 13 days after disease induction. Four days after disease onset, the levels of PBR protein in the spinal cord of the mice are measured by autoradiography and use of ³H-(R)-PK 11195. A decrease in PBR levels after treatment in the treated EAE mice as compared to the levels prior to treatment suggests the potential therapy is useful for the treatment of MS.

Similar experiments are performed to determine effective doses of an MS therapy. EAE is induced in the mice by MOG immunization. The therapy is administered 9 and 13 days after disease induction at varying doses. Four days after disease onset, the levels of PBR in the spinal cord of the mice are measured by autoradiography and use of³H-(R)-PK 11195. A decrease in PBR protein levels in the EAE mice treated with a particular dose of the therapy suggests that dose is useful for the treatment of MS.

A number of embodiments of the invention have been described. Nevertheless, it will be understood that various modifications may be made without departing from the spirit and scope of the invention. Accordingly, other embodiments are within the scope of the following claims.

What is claimed is:

1. A method of evaluating peripheral benzodiazepine receptor (PBR) in a subject, the method comprising:
evaluating a parameter associated with PBR expression in the subject, wherein the subject has multiple sclerosis (MS) and has been administered a TWEAK/TWEAK-R blocking agent.

2. The method of claim 1, further comprising comparing the parameter in the subject to a reference.

3. The method of claim 1, wherein evaluating comprises
evaluating the parameter prior to treatment with a TWEAK/TWEAK-R blocking agent; and
evaluating the parameter after treatment has commenced.

4. The method of claim 3, wherein a decrease in the parameter after treatment has commenced indicates efficacy of the TWEAK/TWEAK-R blocking agent.

5. The method of claim 1, wherein the TWEAK/TWEAK-R blocking agent is selected from the group consisting of: an anti-TWEAK antibody, an anti-TWEAK-R antibody, and a soluble form of the TWEAK receptor.

6. The method of claim 1, wherein the evaluating is performed in vivo.

7. The method of claim 6, wherein the evaluating comprises a PET scan or MRI.

8. The method of claim 7, wherein a region of the spinal cord or brain of the subject is evaluated.

9. The method of claim 6, wherein the evaluating is performed by administering a PBR binding agent to the subject.

10. The method of claim 9, wherein the binding agent is a PBR ligand or an anti-PBR antibody.

11. The method of claim 9, wherein the binding agent is labeled with a detectable label.

12. The method of claim 1, wherein the parameter reflects the amount of PBR binding agent that binds to PBR protein or PBR nucleic acid.

13. The method of claim 1, wherein the evaluating is performed in vitro on a sample obtained from the subject.

14. The method of claim 13 wherein the evaluating comprises quantitative or qualitative assessment of PBR nucleic acid levels.

15. The method of claim 13 wherein the evaluating comprises quantitative or qualitative assessment of PBR protein levels.

16. The method of claim 14 or 15, wherein evaluating is performed by a technique selected from the group consisting of: RT-PCR, Northern blot, ELISA, Western Blot, flow cytometry, and autoradiography.

17. The method of claim 13, wherein the evaluating is performed by contacting a PBR binding agent to the sample.

18. The method of claim 17, wherein the binding agent is a PBR ligand or an anti-PBR antibody.

19. The method of claim 17, wherein the binding agent is labeled with a detectable label.

20. The method of claim 1, wherein the subject is human.

21. A method of evaluating a subject who is being treated for MS, the method comprising:
monitoring a parameter associated with PBR expression in a subject; and
providing a TWEAK/TWEAK-R blocking agent to ameliorate MS to the subject.

22. The method of claim 21, wherein the monitoring comprises evaluating a parameter associated with PBR expression from a subject at least two instances separated by at least 24 hours.

23. The method of claim 21, further comprising comparing the parameter in the subject to a reference.

24. The method of claim 21, wherein monitoring comprises
evaluating the parameter prior to treatment with a TWEAK/TWEAK-R blocking agent; and
evaluating the parameter after treatment has commenced.

25. The method of claim 24, wherein a decrease in the parameter after treatment has commenced indicates efficacy of the TWEAK/TWEAK-R blocking agent.

26. The method of claim 21, wherein the TWEAK/TWEAK-R blocking agent is selected from the group consisting of: an anti-TWEAK antibody, an anti-TWEAK-R antibody, and a soluble form of the TWEAK receptor.

27. The method of claim 21, wherein the monitoring is performed in vivo.

28. The method of claim 27, wherein the monitoring comprises a PET scan or MRI.

29. The method of claim 28, wherein a region of the spinal cord or brain of the subject is evaluated.

30. The method of claim 27, wherein the monitoring is performed by administering a PBR binding agent to the subject.

31. The method of claim 30, wherein the binding agent is a PBR ligand or an anti-PBR antibody.

32. The method of claim 30, wherein the binding agent is labeled with a detectable label.

33. The method of claim 21, wherein the parameter reflects the amount of PBR binding agent that binds to PBR protein or PBR nucleic acid.

34. The method of claim 21, wherein the monitoring is performed in vitro on a sample obtained from the subject.

35. The method of claim 34 wherein the monitoring comprises quantitative or qualitative assessment of PBR nucleic acid levels.

36. The method of claim 34 wherein the monitoring comprises quantitative or qualitative assessment of PBR protein levels.

37. The method of claim 35 or 36, wherein monitoring is performed by a technique selected from the group consisting of: RT-PCR, Northern blot, ELISA, Western Blot, flow cytometry, and autoradiography.

38. The method of claim 34, wherein the monitoring is performed by contacting a PBR binding agent to the sample.

39. The method of claim 38, wherein the binding agent is a PBR ligand or an anti-PBR antibody.

40. The method of claim 38, wherein the binding agent is labeled with a detectable label.

41. The method of claim 21, wherein the subject is human.

42. A method of evaluating a subject who is being treated for MS, the method comprising:
administering, to a subject, a TWEAK/TWEAK-R blocking agent for MS;
before, during, or after administration of the blocking agent, monitoring a parameter associated with PBR expression in the subject; and
comparing results of the evaluation to a reference to provide an assessment of the subject.

43. The method of claim 42, wherein the reference is obtained by a corresponding evaluation of the subject prior to commencing administration of the blocking agent.

44. The method of claim 42, wherein the reference is parameter levels in a control, e.g., a subject that does not have MS or an average value of the parameter in a cohort;
or parameter levels in the subject prior to commencing administration of the blocking agent.

45. The method of claim 42, wherein monitoring comprises
evaluating the parameter prior to administration of a TWEAK/TWEAK-R blocking agent; and
evaluating the parameter after administration has commenced.

46. The method of claim 45, wherein a decrease in the parameter after administration has commenced indicates efficacy of the TWEAK/TWEAK-R blocking agent.

47. The method of claim 42, wherein the TWEAK/TWEAK-R blocking agent is selected from the group consisting of: an anti-TWEAK antibody, an anti-TWEAK-R antibody, and a soluble form of the TWEAK receptor.

48. The method of claim 42, wherein the monitoring is performed in vivo.

49. The method of claim 48, wherein the monitoring comprises a PET scan or MRI.

50. The method of claim 49, wherein a region of the spinal cord or brain of the subject is evaluated.

51. The method of claim 48, wherein the monitoring is performed by administering a PBR binding agent to the subject.

52. The method of claim 51, wherein the binding agent is a PBR ligand or an anti-PBR antibody.

53. The method of claim 51, wherein the binding agent is labeled with a detectable label.

54. The method of claim 42, wherein the parameter reflects the amount of PBR binding agent that binds to PBR protein or PBR nucleic acid.

55. The method of claim 42, wherein the monitoring is performed in vitro on a sample obtained from the subject.

56. The method of claim 55 wherein the monitoring comprises quantitative or qualitative assessment of PBR nucleic acid levels.

57. The method of claim 55 wherein the monitoring comprises quantitative or qualitative assessment of PBR protein levels.

58. The method of claim 56 or 57, wherein monitoring is performed by a technique selected from the group consisting of: RT-PCR, Northern blot, ELISA, Western Blot, flow cytometry, and autoradiography.

59. The method of claim 55, wherein the monitoring is performed by contacting a PBR binding agent to the sample.

60. The method of claim 59, wherein the binding agent is a PBR ligand or an anti-PBR antibody.

61. The method of claim 59, wherein the binding agent is labeled with a detectable label.

62. The method of claim 42, wherein the subject is human.

63. A method of altering the dosage of a TWEAK/TWEAK-R blocking agent, the method comprising
evaluating a parameter associated with PBR expression in a subject, wherein the subject has MS and is being treated with a TWEAKITWEAK-R blocking agent.

64. The method of claim 63, wherein the evaluating comprises
evaluating a parameter of PBR expression in the subject prior to commencing treatment with the TWEAK/TWEAK-R blocking agent; and
evaluating the parameter in the subject after commencing treatment with the TWEAK/TWEAK-R blocking agent,
wherein absence of a decrease in the parameter after treatment has commenced indicates that the dosage of the therapy can be altered.

65. The method of claim 63, further comprising making a treatment decision.

66. The method of claim 63, wherein evaluating comprises
evaluating a parameter of PBR expression in the subject prior to commencing treatment with the TWEAK/TWEAK-R blocking agent; and
evaluating the parameter in the subject after commencing treatment with the TWEAK/TWEAK-R blocking agent,
wherein absence of a decrease in the parameter after treatment has commenced indicates that a second therapy can be administered to the subject.

67. The method of claim 63, wherein evaluating comprises
evaluating the parameter prior to treatment with a TWEAK/TWEAK-R blocking agent; and
evaluating the parameter.

68. The method of claim 67, wherein a decrease in the parameter after treatment has commenced indicates efficacy of the TWEAK/TWEAK-R blocking agent.

69. The method of claim 63, wherein the TWEAK/TWEAK-R blocking agent is selected from the group consisting of: an anti-TWEAK antibody, an anti-TWEAK-R antibody, and a soluble form of the TWEAK receptor.

70. The method of claim 63, wherein the evaluating is performed in vivo.

71. The method of claim 70, wherein the evaluating comprises a PET scan or MRI.

72. The method of claim 71, wherein a region of the spinal cord or brain of the subject is evaluated.

73. The method of claim 70, wherein the evaluating is performed by administering a PBR binding agent to the subject.

74. The method of claim 73, wherein the binding agent is a PBR ligand or an anti-PBR antibody.

75. The method of claim 73, wherein the binding agent is labeled with a detectable label.

76. The method of claim 63, wherein the parameter reflects the amount of PBR binding agent that binds to PBR protein or PBR nucleic acid.

77. The method of claim 63, wherein the evaluating is performed in vitro on a sample obtained from the subject.

78. The method of claim 77 wherein the evaluating comprises quantitative or qualitative assessment of PBR nucleic acid levels.

79. The method of claim 77 wherein the evaluating comprises quantitative or qualitative assessment of PBR protein levels.

80. The method of claim 78 or 79, wherein evaluating is performed by a technique selected from the group consisting of: RT-PCR, Northern blot, ELISA, Western Blot, flow cytometry, and autoradiography.

81. The method of claim 77, wherein the evaluating is performed by contacting a PBR binding agent to the sample.

82. The method of claim 81, wherein the binding agent is a PBR ligand or an anti-PBR antibody.

83. The method of claim 81, wherein the binding agent is labeled with a detectable label.

84. The method of claim 63, wherein the subject is human.

85. A method of evaluating an MS therapeutic, the method comprising:
evaluating a parameter associated with PBR expression in a subject, wherein the subject has MS or experimental autoimmune encephalomyelitis (EAE).

86. The method of claim 85, wherein the therapeutic comprises a TWEAK/TWEAK-R blocking agent.

87. The method of claim 85, further comprising evaluating TWEAK pathway activity in the subject.

88. The method of claim 85, wherein the evaluating comprises
evaluating a parameter of PBR expression in the subject prior to commencing treatment with the therapeutic;
evaluating a parameter of PBR expression in the subject after commencing treatment with the therapeutic; and
wherein absence of a decrease in the parameter after treatment has commenced indicates that the therapeutic is not effective for treating MS or that the dosage of the therapeutic should be altered.

89. The method of claim 85, wherein the evaluating comprises
evaluating a parameter of PBR expression in the subject after commencing treatment with the therapeutic; and
obtaining a reference value for the parameter, wherein a reference value lower than the parameter indicates that the therapeutic is not effective for treating MS or that the dosage of the therapeutic should be altered.

90. The method of claim 85, wherein the reference value is parameter levels in a control, e.g., a subject that does not have MS or an average value of the parameter in a cohort; or parameter levels in the subject prior to commencing treatment with the blocking agent.

91. The method of claim 85, wherein evaluating comprises
evaluating the parameter prior to treatment with a TWEAK/TWEAK-R blocking agent; and
evaluating the parameter after treatment has commenced.

92. The method of claim 91, wherein a decrease in the parameter after treatment has commenced indicates efficacy of the TWEAK/TWEAK-R blocking agent.

93. The method of claim 85, wherein the TWEAKJTWEAK-R blocking agent is selected from the group consisting of: an anti-TWEAK antibody, an anti-TWEAK-R antibody, and a soluble form of the TWEAK receptor.

94. The method of claim 85, wherein the evaluating is performed in vivo.

95. The method of claim 94, wherein the evaluating comprises a PET scan or MRI.

96. The method of claim 95, wherein a region of the spinal cord or brain of the subject is evaluated.

97. The method of claim 94, wherein the evaluating is performed by administering a PBR binding agent to the subject.

98. The method of claim 97, wherein the binding agent is a PBR ligand or an anti-PBR antibody.

99. The method of claim 97, wherein the binding agent is labeled with a detectable label.

100. The method of claim 85, wherein the parameter reflects the amount of PBR binding agent that binds to PBR protein or PBR nucleic acid.

101. The method of claim 85, wherein the evaluating is performed in vitro on a sample obtained from the subject.

102. The method of claim 101 wherein the evaluating comprises quantitative or qualitative assessment of PBR nucleic acid levels.

103. The method of claim 101 wherein the evaluating comprises quantitative or qualitative assessment of PBR protein levels.

104. The method of claim 102 or 103, wherein evaluating is performed by a technique selected from the group consisting of: RT-PCR, Northern blot, ELISA, Western Blot, flow cytometry, and autoradiography.

105. The method of claim 101, wherein the evaluating is performed by contacting a PBR binding agent to the sample.

106. The method of claim 105, wherein the binding agent is a PBR ligand or an anti-PBR antibody.

107. The method of claim 105, wherein the binding agent is labeled with a detectable label.

108. The method of claim 85, wherein the subject is human.

109. A method of evaluating a subject who is being treated for MS, the method comprising:
treating a subject with an anti-TWEAK antibody for MS;
during treatment with the antibody, in vivo monitoring PBR protein levels in the subject, wherein the monitoring comprises administering ¹¹C-labeled PK 11195 to the subject and imaging the subject by use of a PET scan to detect PK 11195 binding to PBR protein; and
comparing results of the monitoring to a corresponding monitoring of the subject prior to commencing treatment with the antibody, wherein a decrease in PK 11195 binding to PBR protein after treatment has commenced indicates efficacy of the antibody.

110. A method of evaluating a subject having, or suspected of having, multiple sclerosis (MS), the method comprising:
evaluating the subject to obtain a parameter associated with one or more genes listed in FIG. 3.

111. The method of claim 110 wherein the subject is being or has been administered a TWEAK/TWEAK-R blocking agent.

112. The method of claim 110 wherein the gene listed in FIG. 3 has a “% reduction by anti-TWEAK relative to Control Ig” shown in FIG. 3 that is greater than 20, 25, 30, or 35.

113. The method of claim 110 wherein the evaluating comprises quantitative or qualitative assessment of nucleic acid levels.

114. The method of claim 110 wherein the evaluating comprises quantitative or qualitative assessment of protein levels.


SEQUENCE LISTING (2 sequences)

SEQ ID NO: 1 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 2 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ